(19) United States
(12) Patent Application Publication
(10) Pub. No.: US 2004/0126900 A1
(43) Pub. Date: Jul. 1, 2004

(54) High affinity peptide- containing nanoparticles

(76) Inventors: Stephen E Barry, Oakland, CA (US); Andrew A Goodwin, Berkeley, CA (US); Dominic Casenas, Fremont, CA (US); Kevin Lindquist, Albany, CA (US); Rachel Decor, Berkeley, CA (US)

Correspondence Address: JACQUELINE S LARSON, P O BOX 2426, SANTA CLARA, CA, 95055-2426, US

(21) Appl. No.: 10/257,455

(22) PCT Filed: Apr. 13, 2001
(86) PCT No.: PCT/US01/12093

(51) Int. Cl. C07K 7/08; C07K 7/06; G01N 33/543

(52) U.S. Cl. 436/523; 530/330; 530/329

(57) ABSTRACT

The present invention is directed to polymeric nanoparticles functionalized with two or more peptide moieties that possess high affinity to biomolecular targets, the peptide moieties being covalently linked to the nanoparticle polymeric core structure, either directly or via a linker molecule. The invention is further directed to methods of synthesizing these polymeric nanoparticles and to the various applications for which they may be used.

### FIELD OF THE INVENTION

The present invention is directed to the field of therapeutic entities. More specifically, this invention relates to particles with desirable therapeutic attributes.

### BACKGROUND OF THE INVENTION

Certain peptides may bind to specific proteins with high affinity and selectivity and can thus be employed in various separation, diagnostic, and therapeutic applications. However, in vivo, peptides are often cleared rapidly from the blood stream. Methods that increase the circulation time of therapeutic peptides, as well as other therapeutic agents, are thus desirable. In one strategy peptides, proteins, and supermolecular complexes such as liposomes have been functionalized with polyethylene glycol (PEG) molecules to increase their circulation time. New therapeutic architectures and delivery methods are desirable.

Increasing the binding strength of therapeutic entities to their intended target is also desirable. In the case of vancomycin, this has been achieved via oligomerization of vancomycin molecules to form oligomers, i.e. dimers (Rao, et.al., Chem. Biol., 6(6):353-9 1999). The dimer shows a substantial increase in binding strength. Because multivalent entities may possess exceptional binding performance, their use is being explored by a number of researchers (S. Borman, Chemical & Engineering News, Oct. 9, 2000, p.48-53).

Peptides such as those containing the arginine-glycine-aspartic acid (RGD) sequence that bind integrin have been attached to alkyl cyanoacrylate nanoparticles for the purpose of enhancing oral delivery of therapeutic molecules physically adsorbed to the nanoparticle surface or dispersed within the nanoparticle (see, EP Pat. 684814). Similarly, nanoparticles have been functionalized with proteins for diagnostic applications.

An important area of current research in therapeutic oncology is focused on the development of antiangiogenic agents, which target tumor vasculature by inhibiting or suppressing blood vessel growth. RGD-containing peptides have been shown to block integrin αᵥβ₃ receptor and rapidly initiate apoptosis of neovascular endothelial cells. Further, they have been shown to inhibit metastasis of several tumor cell lines and tumor-induced angiogenesis. Short cyclic RGD-containing peptides have been developed that are selective for αᵥβ₃ integrin binding (R. Haubner, et al., J. Am. Chem. Soc., 118: 7461-71, 1997). Advantages of small cyclic peptides as therapeutics are their facile synthesis, resistance against proteolysis, and low immunogenicity.

Nanoparticles can be used both to deliver therapeutics and to present high affinity binding elements on their surfaces. Nanoparticles have been formed from the spontaneous aggregation of poly(amino acid) constituents in aqueous solutions (U.S. Pat. No. 5,904,936). The particles thus formed have demonstrated drug delivery uses, the drug being encapsulated within the particles.

The toxicity of certain proteins, such as cytokines, including interleukins, interferons, and tumor necrosis factors, limits their dosage when they are given in a system-wide fashion. However, systemic toxicity can be substantially reduced if the proteins are locally concentrated in the tissue in which their activity is desired. One method to accomplish this is through the creation of fusion proteins, where cytokines are fused to tumor specific antibody domains (Xiang J., Hum Antibodies, 1999, 9, 23-26). The tumor-specific antibodies attach to antigens in the tumor, thereby removing the fusion proteins from system-wide circulation and concentrating them within the tumor. Other methods for concentrating therapeutic proteins to specific tissues would be of value.

Block copolymers may be advantageously used to form nanoparticles for in vivo use. Aluminum porphyrin complexes are known to polymerize a variety of cyclic ethers and esters including epoxide, lactone, and lactide and to make copolymers of epoxides with acid anhydrides or carbon dioxide (Inoue, S., J. Macromol. Sci. 1988, A25, 571). Many of these polymers are generally recognized to possess many of the desirable characteristics of low, if any, toxicity and immunogenicity and are readily cleared from the body. Additionally, the “living” nature of this polymerization system allows the preparation of block copolymers with a narrow molecular weight distribution. Poly(□-caprolactone)co(ethylene oxide) diblock copolymers (Gan, Z.; Jiang, B.; Zhang, J., J. Appl Polym. Sci. 1996, 59, 961) have been shown to readily form nanoparticles and undergo enzymatic degradation (Gan, Z.; Jim, T. F.; Li, M.; Yuer, Z.; Wang, S.; Wu, C., Macromolecules 1999, 32, 590). Nanoparticles have also been formed from the spontaneous aggregation of poly(amino acid) (PAA) block copolymers in aqueous solutions (U.S. Pat. No. 5,904,936). PEG-PAA block copolymers, with the PEG block being hydrophilic and a PAA block comprised of hydrophobic amino acids or amino acid derivatives (such as beta-benzyl-L-aspartate) have also been used to form nanoparticles, and in addition have been investigated for use in the delivery of small molecules (Kwon, G. S.; Naito, M.; Yokoyama, M.; Okano, T.; Sakurai, Y.; Kataoka, K.; Pharm Res 1995, 32, 192; Y. Jeong, J. Cheon, S. Kim, J. Nah, Y. Lee. Y. Sung, T. Akaike, and C. Cho., J. Controlled Release 1998, 51, 169).

Polymerization has been performed in the dispersed phase of microemulsions and reverse microemulsions (for a review, see Antonietti, M.; and Basten, R.; Macromol. Chem. Phys. 1995, 196, 441); for a study of the polymerization of a hydrophilic monomer in the dispersed aqueous phase of a reverse microemulsion, see Holtzscherer, C.; and Candau, F.; Colloids and Surfaces, 1988, 29, 411). Microemulsion polymerization can yield particles in the 5 nm to 50 nm size range.

Modification of nanoparticles such that the nanoparticles themselves display bioactivity may be desirable. Preferably, such nanoparticles should be easy to fabricate, should bind strongly and specifically to the intended molecular targets, should be biocompatible, should be metabolized to nontoxic substances in the body, should be non-immunogenic, and should be designed to avoid undesired clearance from the bloodstream. Circulation time may be affected in part through size. To avoid uptake by the RES system, particles are preferably less than 50 nm. To avoid renal clearance, particles are preferably larger than 5 nm. Also, as mentioned above, circulation time may also be extended through the presence of PEG on the surface of the particles. Targeted delivery of certain therapeutic entities would also be beneficial.

### SUMMARY OF THE INVENTION

This invention is directed to water-soluble polymeric nanoparticles, with each nanoparticle functionalized with two or more peptide moieties that possess high affinity to biomolecular targets, the peptide moieties being covalently linked to the nanoparticle polymeric matrix structure or core. The nanoparticles may optionally further comprise one or more enhancer molecules to facilitate targeting and/or delivery, or they may optionally comprise polyethylene glycol (PEG)-based molecules. The PEG chains may serve as linkers or tethers, with one end attached to the nanoparticle surface and the other end functionalized with a high affinity peptide. The invention is further directed to methods of synthesizing these polymeric nanoparticles and to the various applications for which they may be used. The nanoparticles of the invention disclosed herein are termed Peptide-Functionalized Nanoparticles (PFNs).

The particles of the invention are preferably from about 5 nm to about 1000 nm, more preferably from about 5 nm to about 100 nm, in diameter, and most preferably from 5 to 30 nm. The size of the particles allows their use in vivo as bioactive entities.

The number of high affinity peptide (“HAP”) moieties per nanoparticle can range from 2 to about 1000, preferably from 2 to 100, and most preferably from 2 to 30. The nanoparticles may optionally further be comprised of more than one type of high affinity peptide. As used herein, a peptide “type” is defined as a peptide of a specific molecular structure.

One type of high affinity peptide, herein referred to as “Type 1 peptide”, serves to bind a protein or protein fragment of therapeutic value (referred to herein as a “therapeutic protein”) to the nanoparticle. Another type of high affinity peptide, herein referred to as “Type 2 peptide”, serves to bind the polymeric nanoparticle of the invention to a target protein expressed on the surface of a given cell type or in a certain tissue type.

When compared to peptides alone, the peptide-functionalized nanoparticles disclosed herein may advantageously have longer circulation time. Additionally, the PFNs may bind more strongly to their target, and exhibit lower immunogenicity. The nanoparticles are comprised of biodegradable components that are metabolized to nontoxic substances in mammals.

An additional advantage of the present invention is that multiple high affinity peptide-types with complementary features may be incorporated into a single nanoparticle. This can allow, for instance, for therapeutic nanoparticles to be targeted to a desired cell type, tissue, or organ using one or more than one targeting peptides; or a therapeutic protein to be delivered to a desired target site.

In one embodiment of the invention, the nanoparticle cores are composed of hydrophobic/hydrophilic block copolymers. The block copolymers employed will have the property of spontaneously forming nanoparticles in aqueous systems under certain conditions. Preferably the block copolymer chains are comprised of at least two types of recurring monomers. The preferable block forms are of the AB or ABA type. “A Blocks” are hydrophilic, for example polyethylene glycol or polyalkylene oxide. “B Blocks” are hydrophobic and neutral, for example polycaprolactone. Block copolymers with a narrow molecular weight distribution are preferred. Because of their degradability, biocompatibility, well-documented synthesis, tailorability of the chemical nature, and the ease of which they may be crosslinked, poly(amino acids) (“PAAs”) are preferred as hydrophobic building blocks in the present invention. They may be employed in forming the hydrophilic block as well.

In another embodiment of the invention, the nanoparticle cores are composed of crosslinked hydrophilic building blocks. These nanoparticles are fabricated by first forming a nanoparticle core through the crosslinking of the building blocks in the dispersed aqeuous phase of a reverse microemulsion. The crosslinkable moieties are preferably acrylates or acrylamides. Carbohydrate derivatives are preferably used as building blocks.

One embodiment of the invention is directed to a method for the molecular recognition of biomolecular targets. More particularly, this embodiment is directed to nanoparticles comprised of Type 2 peptide sequences that possess high affinity to certain biomolecular targets, the peptide being covalently linked to hydrophilic polymer or block copolymer nanoparticle matrix molecules.

In another embodiment of the invention, protein therapeutics, including hydrophilic proteins, are incorporated into the nanoparticle structures for drug delivery applications. More particularly, this embodiment is directed to nanoparticle medicines that controllably release therapeutic proteins. In this embodiment the nanoparticles are comprised of three types of molecular structures: nanoparticle matrix molecules, Type 1 peptide sequences that possess high affinity to therapeutic proteins, and the therapeutic proteins. The therapeutic protein-peptide affinity allows the retention of the therapeutic protein in the nanoparticle in use, extending circulation time of the protein. One or more peptide types capable of high affinity binding to the therapeutic protein may be used.

A further embodiment of the invention provides a method for the controlled delivery of therapeutic proteins to the vicinity of the targeted cell or tissue type. The nanoparticles of this embodiment are comprised of four types of molecular structures: nanoparticle matrix molecules, Type 1 short peptide sequences that possess high affinity to therapeutic proteins, Type 2 short peptide sequences that possess high affinity to proteins expressed on certain cells or in certain tissues, and therapeutic proteins. Thus, when administered as a therapeutic, the nanoparticles containing the therapeutic protein will concentrate in tissue or on cell surfaces expressing a targeted protein.

### DETAILED DESCRIPTION OF THE INVENTION

The terms “a” and “an” mean “one or more” when used herein.

By “water-soluble” is meant, herein and in the appended claims, having a solubility in water of greater that 10 mg/mL, and preferably greater than 50 mg/mL.

Nanoparticles comprised of high affinity molecules are provided; the nanoparticles are water-soluble and capable of in vivo delivery. More particularly, each polymeric nanoparticle is functionalized with two or more peptide moieties that possess high affinity to biomolecular targets, the peptide moieties being covalently linked to the nanoparticle polymeric matrix structure. The invention is further directed to methods of synthesizing these polymeric nanoparticles and to the various applications for which they may be used.

The nanoparticles of the invention may range in size from about 5 nm to about 1000 nm, more preferably from about 5 nm to about 100 nm in diameter, and most preferably about 5 nm to about 30 nm. Nanoparticles in the 5 to 30 nm size range may effectively avoid renal clearance and uptake by the reticuloendothelial system (RES). Additionally, small particles can advantageously exit the blood stream to reach desired cell, tissue, or organ targets.

The number of HAP moieties per nanoparticle can range from 2 to about 1000, preferably from 2 to 100, and most preferably from 2 to 30. The nanoparticles may optionally further be comprised of more than one type of high affinity peptide. As used herein, a peptide “type” is defined as a specific molecular structure.

Peptides used as high affinity building blocks according to this invention will generally possess binding affinities between 10⁻⁴ and 10⁻⁹ M. The high affinity peptides may be comprised of known peptide ligands to receptors of interest. For instance, Phoenix Peptides' peptide ligand-receptor library (http://www.phoenixpeptide.com/Peptidelibrarylist.htm) contains thousands of known peptide ligands to receptors of potential therapeutic value. The HAPs may be natural peptides such as, for example, lactams, dalargin and other enkaphalins, endorphins, angiotensin II, gonadotropin releasing hormone, thrombin receptor fragment, myelin, and antigenic peptides. High affinity peptide building blocks useful in this invention may be discovered via high throughput screening of peptide libraries (e.g. phage display libraries or libraries of linear sequences displayed on beads) to a protein of interest. Such screening methods are known in the art (for example, see C. F. Barbas, D. R. Burton, J. K. Scott, G. J. Silverman, Phage Display, 2001, Cold Spring Harbor Laboratory Press, Cold Spring Harbor, N.Y.). The high affinity peptides may be comprised of modified amino acids or completely synthetic amino acids.

The length of the recognition portion of the high affinity peptide can vary from about 3 to about 100 amino acids. Preferably, the recognition portion of the peptide ranges from about 3 to about 15 amino acids, and more preferably from 3 to 10 amino acids. Shorter sequences are preferred because peptides of less than 15 amino acids may be less immunogenic compared to longer peptide sequences. Small peptides have the additional advantage that their libraries can be rapidly screened. Also, they may be more easily synthesized using solid state techniques.

The use of multiple high affinity peptide molecules of the same molecular structure or of different molecular structure to make up the nanoparticle can increase the avidity of the nanoparticle. As used in the present invention, “high affinity” means a binding of a single peptide to a single target molecule with a binding constant stronger than 10⁻⁴ M, while “avidity” means the binding of two or more such peptide units to two or more target molecules on a cell or molecular complex.

Reagents and starting materials in some embodiments can be obtained commercially. For example, amino acids can be purchased from chemical distributors such as Sigma-Aldrich (Milwaukee, Wis.), Pierce Chemical Company (Rockford, Ill.), and Chemdex (www.chemdex.com). Additionally, chemical product directories and resources, such as http://pubs.acs.org/chemcy/, may be used to locate starting materials. Peptides to be used as high affinity binders can be purchased from many sources, one being Peptide Biosynthesis (www.peptidebiosynthesis.com).

The nanoparticle cores are chosen to be readily synthesized, degradable within the body on a desired time scale, to be nontoxic, to allow facile functionalization with high affinity peptides, and to allow the inclusion of proteins of therapeutic value and other therapeutic substances.

I. PFNs Comprised of Block Copolymers

Where the nanoparticle core is comprised of block copolymers, the block copolymers will be comprised of hydrophobic blocks and hydrophilic blocks. Polymeric materials with this structure are known to be amenable to the formation of nanoparticles with the structures preferred for use in this invention. The hydrophobic and hydrophilic blocks will be comprised of materials which are known to be nontoxic, nonimmunogenic, are eliminated by degradation and clearance mechanisms within the body (for a review of polymers with these properties see Amass, W., Amass, A., Tighe, B., Polymer International, 1998, 47, 89), and also allow for the preparation of nanoparticles that are readily functionalized with a peptide, protein or pharmaceutical agent. The advancements, improvements, and modifications to the prior art that are necessary to synthesize these materials for the preparation of peptide-functionalized nanoparticies are described herein.

In one embodiment, the hydrophobic/hydrophilic block copolymers to be used in this invention can be synthesized using aluminum porphyrin chemistry largely developed by Inoue (see Inoue, S., J. Macromol. Sci., 1988, A25, 571; and references cited therein for a review of aluminum porphyrin living polymerizations). Aluminum porphyrin complexes are known to polymerize a variety of cyclic ethers and esters including epoxide, lactone, and lactide and to make copolymers of epoxides with acid anhydrides or carbon dioxide (Inoue, ibid.). Many of these polymers are generally recognized to posses many of the desirable characteristics of low, if any, toxicity and immunogenicity and are readily cleared from the body. Because of the “living” nature of this polymerization system, block copolymers can be prepared.

As one example of synthesis using aluminum porphyrin chemistry, poly(ε-caprolactone)co(ethylene oxide) diblock copolymer can be prepared as shown in Reaction Scheme 1 (Gan, Z.; Jiang, B.; Zhang, J., J. Appl. Polym. Sci., 1996, 59, 961) and readily form nanoparticles that undergo enzymatic degradation (Gan, Z.; Jim, T. F.; Li, M.; Yuer, Z.; Wang, S.; Wu, C., Macromolecules, 1999, 32, 590). PEG is a preferred hydrophilic block because of PEG's abilities to increase the circulation time of entities to which it is attached and to impede interaction of immune system components with the PEGylated entities are well documented in the art.

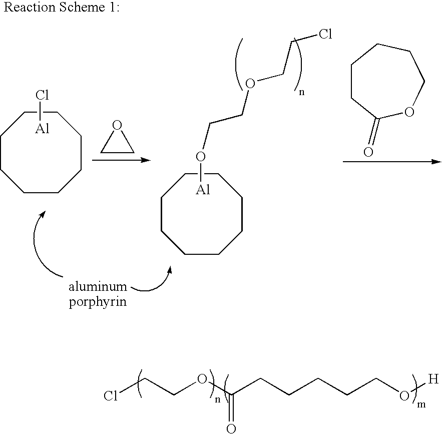

The successful synthesis of a poly(ε-caprolactone)co(ethylene oxide) diblock copolymer serves as useful precedent for the preparation of additional copolymers that can be used in the preparation of peptide-functionalized nanoparticles. Substitution of ε-caprolactone by propane oxide, 1,2-butene oxide, 3,6-dimethyl-1,4-dioxane-2,5-dione (lactide), or a mixture of ethylene oxide and succinic anhydride provides routes to the preparation of new hydrophilic/hydrophobic block copolymers (Reaction Scheme 2).

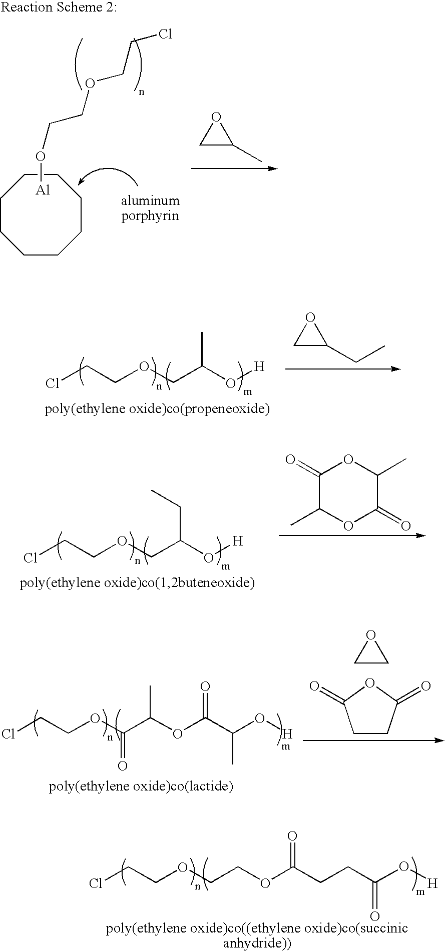

Once the hydrophobic/hydrophilic block copolymers are synthesized, they can be modified by techniques known to those familiar with the art of organic synthesis to include functional groups that readily couple to active agents; that is, to peptides, proteins, or derivatized peptides and protein structures, as well as to pharmaceutical compounds. The coupling reaction is designed to occur at specific points on the active agent by making use of chemoselective ligation reactions. The reacting moieties preferentially react with one another rather than with other moieties such as amines, alcohols and carboxyls typically found on a peptide or protein. A variety of chemoselective reactions have been identified and have been recently reviewed (Lemieux, G. A.; Bertozzi, C. R., Trends in Biotechnology, 1998, 16, 506), and many of these strategies may be employed in the current invention. As an example that demonstrates the utility of one of several possible chemoselective ligation reactions, the terminal chloride located on the first block of the polymers described in the above schemes is readily converted to a thiol by reaction with sodium sulfhydride (March, J. Advanced Organic Chemistry 4^{th} Edition, John Wiley & Sons, Inc. New York, 1992, pp. 406-407) (Reaction Scheme 3).

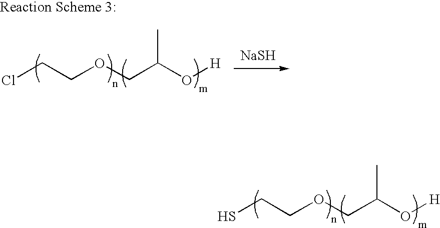

After isolation, the thiol-containing polymer is coupled to the peptide by the highly selective reaction of thiols with the α-bromo carbonyl functional group (Reaction Scheme 4) (see Muir, T. W.; Williams, M. J.; Ginsberg, W. H.; Kent, S. B. H., Biochemistry, 1994, 33, 7701, for an example), a functionality that is readily introduced to lysine-containing peptides (see Dawson, P. E.; Kent, S. B. H., J. Am. Chem. Soc., 1993, 115, 7263 for an example).

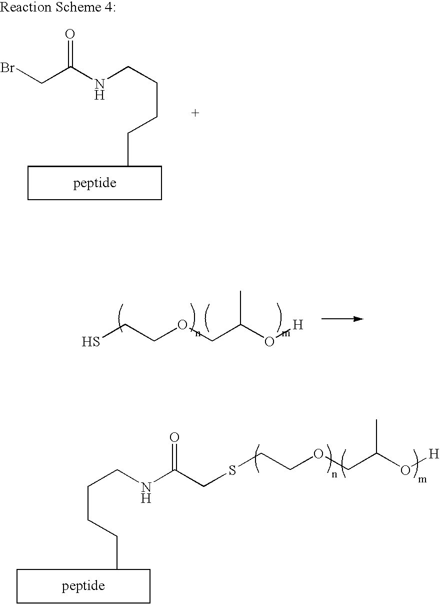

Additionally, functionalities which may be crosslinked after particle formation may be included. Preferably, the crosslinkable moiety is included in the hydrophobic block to decrease the occurrence of interparticle crosslinking. For example, inclusion of a small amount of 1,2-epoxy-4-pentanone ethylene ketal in the polymerization solution during formation of the hydrophobic block will incorporate some cyclic ketals in the hydrophobic block. These ketals may then be cleaved on exposure to acidic water, regenerating the ketone functionality. The ketone functionality can then be crosslinked by reaction with a di(amino-oxy) containing compound such as that made from ethylene diamine (Scheme 5).

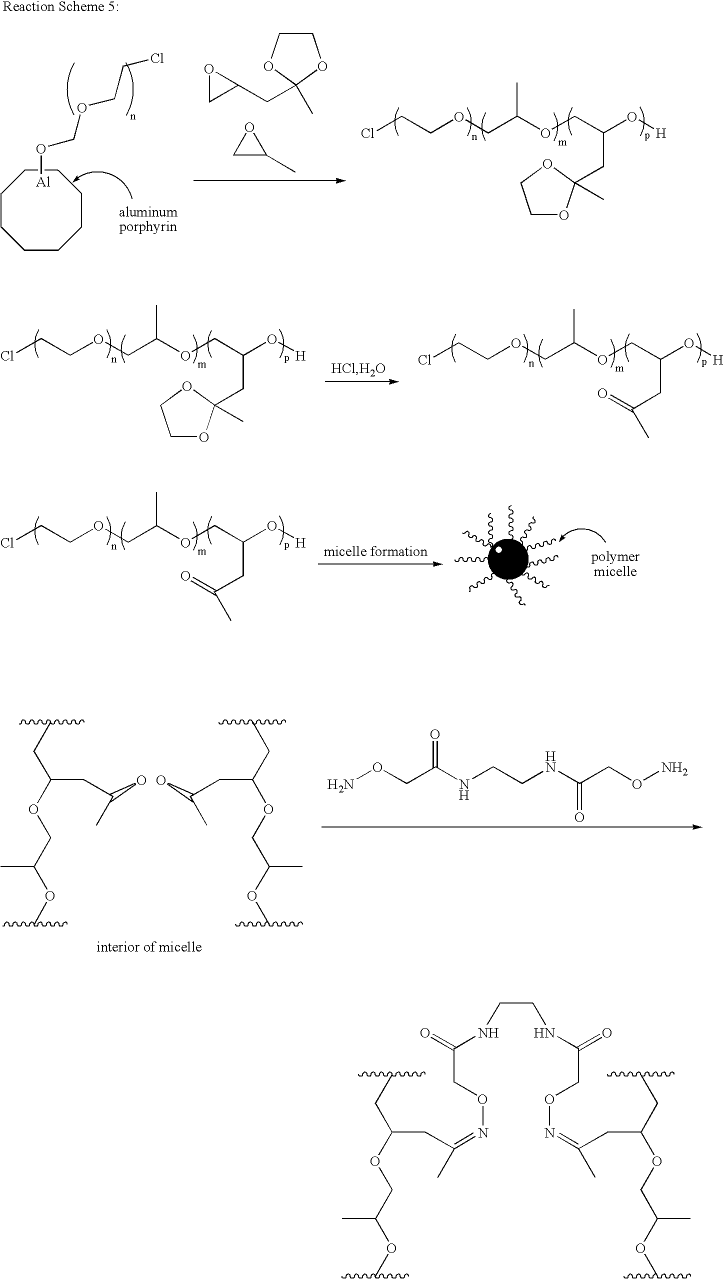

Novel polymers made from the aluminum porphyrin living polymerization of epoxides or cyclic esters from a carbohydrate “block” (Reaction Scheme 6) can be employed. These block copolymers will have similar properties to dextran/styrene or dextran/acrylate block copolymers, the synthesis of which is discussed in Haddleton, D. M.; Ohno, K., Biomacromolecules, 2000, 1, 152. However, the dextran/epoxide or dextran/cyclic ester have the added advantage of being composed of polymer segments that are generally accepted as being non-toxic and able to break down into non-toxic degradation products that are eliminated from the body.

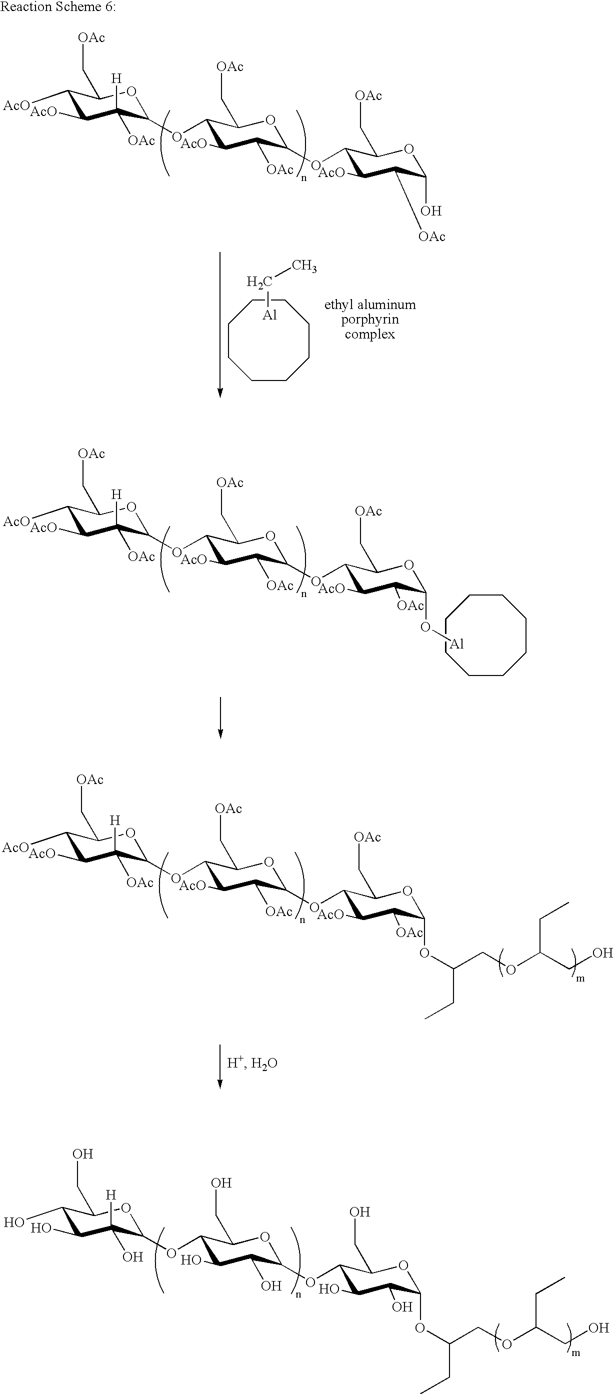

While the above dextran copolymers are capable of forming the nano particles at this point, for use in this invention they must first be modifieds in order to undergo chemoselevtive ligation to the high affinity peptide or to a PEG linker, or to facilitate crosslinking of block copolymer chains. One method of accomplishing this is through the series of reactions outlined in Reaction Scheme 7. The copolymer is first reacted with 1,1′-carbonyldiimidazole, followed by the addition of ethylene diamine to introduce the amine functionality to the dextran block. Further modification with bromo acetic acid anhydride leaves the α-bromo carbonyl group on the dextran. As before, this group can be chemoselectively ligated to a thiol, such as the side chain of a cysteine residue of the peptide or a thiol-terminated PEG. Those familiar with the arts of organic synthesis, peptide synthesis, or biochemistry will recognize that additional functionalities may be used for the ligation step such as, but not limited to, hydrazides, aminooxy, thiosemicarbazide, α-amino thiols, n-hydroxysuccinimide activated esters, halogens, tosylates, and carbodiimide-assisted ester and amide formations.

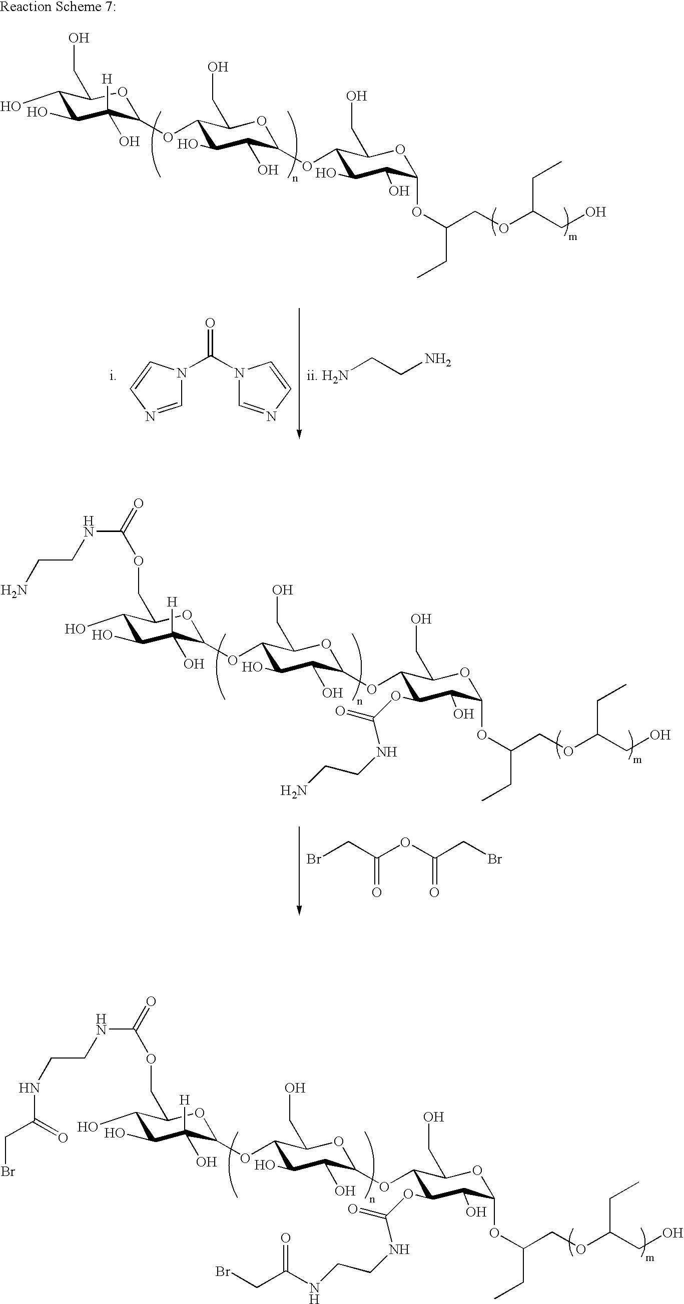

The modified block copolymer will react with the thiol-containing molecule as shown in Reaction Scheme 4.

As with PEG-polycaprolactone blocks, functionalities which may be crosslinked after particle formation may be included. Preferably, the crosslinkable moiety is included in the hydrophobic block to decrease the occurrence of interparticle crosslinking. This can be accomplished, for example, by inclusion of 1,2-epoxy-4-pentanone ethylene ketal in the polymerization mixture, deprotection, and crosslinking with a di(amino-oxy) containing compound as described above.

Because of their degradability, biocompatibility, well-documented synthesis, tailorability of the chemical nature, and the ease of which they may be crosslinked, poly(amino acids) (“PAAs”) are preferred as hydrophobic building blocks in the present invention. Additionally, they may be employed in forming the hydrophilic block as well.

The PAA cores may be synthesized using standard amino acid linking methods discussed in, for example, U.S. Pat. No. 5,904,936. However, the polymerization method employed in '936 may yield broad molecular weight distributions and poorly understood structures (Deming, T. J., Nature 1997, 390, 386-389; Deming, T. J., J. Am. Chem. Soc. 1997, 119, 2759-2760; and references cited in those articles). For example, as discussed in '936, a block structure may be desirable because it forms colloidal particles at lower molecular weights. While not wishing to be bound by theory, the polymerization method used in '936 may produce chains that do not have such a well-defined block structure but rather a complex mixture of polymeric species, and such structures may form less stable colloidal particles. Thus, it may be advantageous to synthesize the PAA chains via a controlled polymerization scheme using the recently developed living polymerization methods developed by Deming (supra Deming; Deming, T. J., Macromolecules 1999, 32, 4500-4502) to isolate well-characterized PAAs with a discrete structure.

PAA hydrophobic block and PEG hydrophilic block: In this embodiment, the PAA block is comprised of hydrophobic amino acids and their derivatives, such as Leu, Ile, Val, Ala, Tyr, Phe, and beta-benzyl-L-aspartate. PEG can be attached to the block using several methods. For example, PEG may be attached to the PAA (either the carboxylic acid or amine terminus) via carbodiimide coupling reactions familiar to those skilled in the art of peptide synthesis (G. T. Hermanson, Bioconjugate Techniques, Academic Press, San Diego, 1996). The PEG may be attached to the PAA while it is on a resin used in solid state peptide synthesis, or after the PAA is cleaved from the resin. Alternatively, the use of transition metal-mediated polymerizations of α-amino acid-N-carboxyanhydrides (NCAs) allows for the end-group functionalization of PAAs during their synthesis (Curtin, S. A., Deming, T. J., J. Am. Chem. Soc., 1999, 121, 7427-7428). This technology can be adapted to assist in the synthesis of PAA-PEG compounds by using a PEG-functionalized initiating ligand on the transition metal complex. The PEG functionalized initiating ligand would start the polymerization of the NCAs, and would remain covalently attached to the PAA chain, giving the desired block architecture.

PAA hydrophobic and PAA hydrophilic blocks: In this embodiment, the poly(amino acid) chains (“PAA”) are comprised of blocks of hydrophobic and neutral amino acids, such as Leu, Ile, Val, Ala, Tyr and Phe, and hydrophilic amino acids such as Glu, Asp, Lys, Arg, and His. Preferably, the ionic amino acid has a side chain with a carboxyl functionality (Glu, Asp). It is preferable to limit the type of amino acids in each block to one or two peptides. For instance, the hydrophobic block may be composed entirely of leucine, or it may be a random copolymer of leucine and valine. Most preferably, the hydrophilic block is composed entirely of Glu, while the hydrophobic block is a copolymer of Leu and Val. The PAAs are preferably of block form, for example, of the AB or ABA type.

PAA Crosslinking: After spontaneous formation, the nanoparticle structure may be further stabilized by crosslinking the block copolymer chains. Such crosslinking can be accomplished in several ways generally known to those of skill in the polymeric art. One method is to include amine residues in the chain structure, such as for example lysine residues in the PAA structure. The percentage of amine residues should be low enough that the ability to form nanoparticles is not reduced beyond an acceptable degree. The amine residues can then be functionalized, for example with an acrylate moiety by using the reagent N-hydroxy-succinimideacrylate. Subsequent to spontaneous nanoparticle formation, the acrylate functionalities from different chains within the same nanoparticle can be crosslinked together using standard free-radical polymerization techniques. Alternatively, glutaraldehyde or other reagents well known to those in the art (see, for example, G Hermanson, Bioconjugation Techniques, Academic Press, San Diego, 1996) may also be used to crosslink amine residues included in the chains. As with linking HAP to block copolymers, crosslinking of block copolymer chains to one another can also be accomplished using chemoselective binding pairs (Lemieux, G. A.; Bertozzi, C. R., Trends in Biotechnology, 1998, 16, 506). Chemoselective reaction pairs, in which the reactants have low or no reactivity towards the moieties of the HAPs or the proteins are preferred.

Preferably crosslinking is performed in the hydrophobic block, away from the surface of the nanoparticle to reduce interparticle reactions. When using chemoselective reaction pairs, block copolymer chains can be crosslinked through ketone groups included on the block copolymer via levulinic acid-functionalized amine termini or lysine residues (dispersed in the hydrophobic block during synthesis for this purpose) and amino-oxy groups.

Peptide Attachment: Type 2 high affinity peptides should be located at or near the surface of the resulting nanoparticle for the highest degree of biological activity. To locate the peptide near the surface of the particle, the HAP is preferably attached to one or both ends of the block copolymer chain. Alternatively, HAPs may be incorporated by attaching HAP molecules to one or more residues along the block copolymer chain. In one embodiment, the architecture can be of the ABA type, with the B block comprised of the hydrophobic polymer, the A blocks comprised of the hydrophilic polymer, and the high affinity peptides attached to the ends of the hydrophilic polymer blocks. Alternatively, a high density of peptides may be incorporated by attachment to multiple residues or intermediate molecules along the block copolymer chain. Preferably, the HAPs are attached to the free terminus of the hydrophilic block.

HAP molecules can be attached directly to block copolymer molecules or via PEG-block copolymer molecules prior to nanoparticle formation, or after nanoparticles have been formed. While not wishing to be bound by theory, when HAPs are attached to the copolymer chains prior to nanoparticle formation, short HAP sequences of 3 to 10 amino acids are preferred as opposed to longer sequences, to reduce the potential of disrupting the spontaneous, thermodynamically-driven formation of colloidal dispersions of the copolymers. When the nanoparticle is comprised of PEG chains, the peptide may be attached to the free end of the PEG molecule before, during, or after particle formation.

Additionally, coupling of HAPs to the block copolymer can be accomplished via attachment to copolymer side chains using standard coupling techniques known to those familiar with peptide synthesis (Jones, J., supra; Bodanszky, M, Bodanszky, A., supra). For example, this can be accomplished by coupling the amine terminus of the HAP to the carboxylic acid side chains of the Glu or Asp residues of a PAA. Conversely, the carboxylic acid terminus of the HAP may be covalently attached to the nitrogen-containing functionalities of Lys, Arg, or His in the PAA. Here, the carboxylic acid terminus of either the PAA or the high affinity peptide is converted to an activated ester (with DCC or other reagents), and condensed with the amine functionality of the reaction partner.

The block copolymers may also be modified by techniques known to those familiar with the art of organic synthesis, to include functional groups that enable chemoselective ligation reactions (Lemieux, ibid.). The reacting moieties preferentially react with one another rather than with other moieties such as amines, alcohols and carboxyls typically found on a peptide or protein. As an example that demonstrates the utility of one of several possible chemoselective ligation reactions, a thiol-containing functionality on a PAA, which can be included by incorporation of a cysteine residue, or a thiol-terminated PEG is coupled to the peptide by the highly selective reaction of thiols with the α-bromo carbonyl functional group (see Muir, T. W.; Williams, M. J.; Ginsberg, W. H.; Kent, S. B. H. Biochemistry, 1994, 33, 7701 for an example), a functionality that is readily introduced to lysine-containing peptides or proteins (see Dawson, P. E.; Kent, S. B. H. J. Am. Chem. Soc., 1993, 115, 7263 for an example).

PEG may be used as the hydrophilic block in the block copoymers used to create the nanoparticles. Alternatively, the block copolymers used to create the nanoparticles may consist of other polymers, for instance a hydrophilic PAA block may be used. In the latter case, after nanoparticle formation, because of the benefits of PEGylation well documented in the literature, it may be beneficial to functionalize the nanoparticle surface with PEG chains. Bifunctional PEG chains can be used to link the high affinity peptides to the surface, or monofunctional PEG chains may be used to modify the surface, as discussed below.

The PEG chains are preferentially hetero- or homo-difunctionalized, with molecular weights between about 1,000 and about 20,000 molecular weight average.

For this use, bifunctional PEGs that are terminated at both ends with the same reactive moieties or heterobifunctional PEGs that are terminated with two different reactive moieties can be used. Representative reactive groups that can be used as terminuses on the PEG include, but are not limited to, amines, aldehydes, f-halo carbonyl, amino-oxy, hydrazide, thiols, ketones, halids, and epoxides. In the case of bifunctional PEGS, two identical groups are located on each end of the PEG. Heterobifunctional PEGs have one unique group located at each end of the PEG. Attachment of PEG to the nanoparticle may then be accomplished, for example, by coupling the carboxy terminus of a copolymer chain to the amine terminus of a PEG chain using a carbodiimide such as EDC (G. T. Hermanson, Bioconjugate Techniques, Academic Press, San Diego, 1996). After PEG attachment to the nanoparticles, the free, reactive terminus of a portion of the PEG chains can be reacted with HAP molecules.

The HAP peptide can be attached to one PEG terminus and subsequently the HAP-PEG molecule can be attached to PAA or to the nanoparticle through the other PEG terminus. The HAP may be formed in its entirety prior to attachment to PEG, or it may be formed by the sequential attachment of single amino acids to a PEG terminus. Using the latter method, the PEG end that will be attached to the nanoparticle can initially be bound to a resin used for solid phase peptide synthesis. For example, di-amino PEG may be used as the starting material. This polymer may be coupled to a variety of resins that may be found to be appropriate for this procedure, for example PEGA resin (for an example of PEG-peptide conjugation, see Auzanneau, F. I.; Meldal, M.; Bock, K.; J. Pept Sci. 1991, 1, 31). The HAP is then grown off of the unreacted PEG terminus using standard reagents and procedures used in solid phase peptide synthesis. Preferably, these procedures make use of FMOC-or BOC-based protecting group strategies, and use DCC, or other carboxylic acid activating agents, for the preparation of the poly amide backbone of the peptide. Upon completion of the sequence, the PEG is cleaved from the substrate resin, regenerating the PEG terminus that was used to attach the PEG to the resin. The exact nature of the cleaving agent will be determined by other functionalities present in the peptide and the identity of the substrate resin. In the preferred example outlined above in which a trityl-functionalized resin is used, cleavage is accomplished by using TFA in methylene chloride. Once liberated, the terminus may undergo subsequent modification, if necessary, to a desired functionality which will assist in attachment to the nanoparticle. For example, coupling to BOC-amino-oxy acetic acid will, after removal of the BOC protecting group with TFA, yield an amino-oxy-functionalized PEG terminus. The amino-oxy moiety may then be reacted with aldehyde or ketone groups present on the block copolymer chains or the nanoparticle. Protecting groups, used as an aid during synthesis of the peptide, may be removed at several stages during the synthesis, with the exact procedural location of their removal dictated by the overall synthetic strategy employed. Similarly, peptide side group modification may also occur at several points during the synthesis.

Once the desired PEG-peptide species has been synthesized with the terminus functionality present, it is attached to the nanoparticle. To assist in this coupling procedure, complimentary reactive pairs will be pre-selected (one as the PEG terminus of the PEG-peptide complex, the other as a functionalized building block, which is included in the nanoparticle formulation). For example, an amino-oxy group may be placed on the PEG terminus and a ketone or aldehyde building block, or an amino acid synthesized with a ketone or aldehyde functional group, will be incorporated into the nanoparticle structure. These two functionalities are known to react in high yields with one another preferentially (Lemieux, ibid.) and thus will couple the PEG-peptide complex to the nanoparticle. Other known reactive pairs, some less selective than that discussed, may also be used, with the exact choice being based on synthetic, toxicological, economic, and other considerations.

PEG Functionalization to Increase Circulation: In one embodiment of the invention polyethylene glycol (PEG) or other polyalkylene oxides (PAO) are attached to the block copolymer chains prior to or after nanoparticle formation such that they are located at the surface of the nanoparticle. If the PEGs are attached prior to nanoparticle formation, their presence must not interfere with spontaneous nanoparticle formation. Such interference may result if the PEG attachment makes the PEG-block copolymer molecules too hydrophilic. Thus, in the case where the PEG chains are attached prior to nanoparticle formation, the molecular weights of the PEG chains and the hydrophilic copolymer blocks should be kept low enough so that nanoparticle formation spontaneously occurs under the appropriate aqueous solution conditions. If the PEG chains are attached after nanoparticle formation, care must be taken that such attachment does not result in the disruption of the nanoparticle structure. One method that can be used to ensure that this does not occur is to crosslink the copolymer chains within the nanoparticle structure prior to PEG molecule attachment.

PEG used for the purpose of increasing circulation time is not required to present a HAP at its terminus. Thus, these PEG chains may be monofunctional; that is, one terminus is functionalized with a reactive group such as an amine, allowing the PEG chain to be attached to a surface or a molecule through this moiety, while the other terminus is generally inert at physiological conditions, for example a methoxy group. Alternatively, bifunctional PEG that is attached at one terminus to the block copolymer chains or nanoparticle surface can be used to increase circulation time. In this case, it is preferable that the free end, that is, the end not attached to the copolymer or nanoparticle, is “capped” or made essentially non-reactive, for example with a methoxy group using routine methods known in the art.

As practiced in the invention, whether PEG is used as a linker or to increase circulation time, the PEG chains are linear, with the preferred molecular weight of the individual PEG chains ranging in molecular weight from 1000 to 50,000 Da.

Nanoparticle Formation from Block Copolymers: Once the peptide-functionalized block copolymer has been synthesized, nanoparticles for targeted delivery can be formed as follows. Block copolymers, a percentage of which may be functionalized with Type 2 peptide-functionalized block copolymers, are added to an aqueous buffer solution. For example, a dilute solution of the Type 2 peptide/block copolymer is prepared in a solution containing a water-soluble organic solvent, such as DMF, THF, and the like. The water-soluble organic solvent is then removed from the water by methods such as dialysis or distillation at reduced pressure.

II. PFNs Comprised of Crosslinked Hydrophilic Polymers

In one embodiment of the invention, hydrophilic building blocks with polymerizable groups are employed to form stable nanoparticle cores. The building blocks are crosslinked in the dispersed aqueous phase of reverse microemulsions. The number of polymerizable groups attached to one single building block can range, for example, from about one to three for low molecular weight building blocks, to ten or more for polymeric building blocks. Building blocks that contain more than one polymerizable group can act as crosslinking agents and enable the formation of a gel network. Using different amounts and proportions of building blocks from a set of building blocks with one, two, or more polymerizable groups allows formation of polymer networks of different compliancy upon polymerization.

Exemplary crosslinkable groups include, but are not limited to, acrylate, acrylamide, vinyl ether, styryl, epoxide, maleic acid derivative, diene, substituted diene, thiol, alcohol, amine, hydroxyamine, carboxylic acid, carboxylic anhydride, carboxylic acid halide, aldehyde, ketone, isocyanate, succinimide, carboxylic acid hydrazide, glycidyl ether, siloxane, alkoxysilane, alkyne, azide, 2′-pyridyidithiol, phenylglyoxal, iodo, maleimide, imidoester, dibromopropionate, and iodacetyl.

Free Radical Polymerization: Preferred polymerizable functionalities are acrylate and acrylamide moieties. Such moieties are amenable to free-radical polymerization. Free-radical polymerization can be readily achieved through the combination of UV light and photoinitiators, redox-coupled free-radical initiators, or heat and heat-activated initiators.

Exemplary monomeric building blocks used to form the nanoparticle core include acrylamide, sodium acrylate, methylene bisacrylamide, ammonium 2,2-bisacrylamidoacetate, 2-acrylamidoglycolic acid, 2-aminoethyl methacrylate, ornithine mono-acrylamide, ornithine diacrylamide sodium salt, N-acryloyltris(hydroxymethyl)-methylamine, hydroxyethylacrylate, N-(2-hydroxypropyl)-acrylamide, 2-sulfoethylmeth-acrylate, 2-methacryloylethyl glucoside, glucose monoacrylate, glucose-1-(N-methyl)-acrylamide, glucose-2-acrylamide, glucose-1,2-diacrylamide, maltose-1-acrylamide, sorbitol monoacrylate, sorbitol diacrylate, sucrose diacrylate, sucrose mono(ethylenediamine acrylamide), sucrose di(ethylenediamine acrylamide), sucrose di(diethylenetriamine acrylamide), kanamycin tetraacrylamide, kanamycin diacrylamide, dextran multiacrylamide, sucrose mono(ethylenediamine acrylamide) mono(diethylenetriamine acrylamide) mono(phenyl alanine) sodium salt, as well as other acrylate- or acrylamide-derivatized sugars.

In a preferred embodiment, at least some of the building blocks are carbohydrates. In the case of carbohydrate building blocks, the carbohydrate region is usually comprised of a plurality of hydroxyl groups, wherein at least one hydroxyl group is modified to include at least one polymerizable group.

The carbohydrate region of the carbohydrate building block may include a carbohydrate or carbohydrate derivative. For example, the carbohydrate region may be derived from a simple sugar, such as glucose, ribose, arabinose, xylose, lyxose, allose, altrose, mannose, gulose, idose, galactose, fructose or talose; a disaccharide, such as maltose, sucrose, lactose, or trehalose; a trisaccharide; a polysaccharide, such as cellulose, starch, glycogen, alginates, inulin, and dextran; or modified polysaccharides. Other representative carbohydrates include sorbitan, sorbitol, chitosan and glucosamine. The carbohydrate may include amine groups in addition to hydroxyl groups, and the amine or hydroxyl groups can be modified, or replaced, to include a crosslinking group, other functionalities, or combinations thereof.

Carbohydrate-based building blocks may be prepared from the carbohydrate precursor (e.g. sucrose, sorbital, dextran, etc.) by standard coupling technologies known in the art of bioorganic chemistry (see, for example, G Hermanson, Bioconjugation Techniques, Academic Press, San Diego, 1996, pp 27-40,155,183-185, 615-617; and S. Hanesian, Preparative Carbohydrate Chemistry, Marcel Dekker, New York, 1997.) For example, a crosslinkable group is readily attached to a carbohydrate via the dropwise addition of acryloyl chloride to an amine-functionalized sugar. Amine-functionalized sugars can be prepared by the reaction of ethylene diamine (or other amines) with 1,1′-carbonyldiimidazole-activated sugars. Other reactions that introduce an amine on the carbohydrate may also be used, many of which are outlined in Bioconjugation Techniques (supra).

Carbohydrate-based building blocks may also be prepared by the partial (or complete) functionalization of the carbohydrate with moieties that are known to polymerize under free radical conditions. For example, methacrylic esters may be placed on a carbohydrate at varying substitution levels by the reaction of the carbohydrate with methacrylic anhydride or glycidyl methacrylate (Vervoort, L.; Van den Mooter, G.; Augustijins, P.; Kinget, R. International Journal of Pharmaceutics, 1998, 172, 127-135).

Carbohydrate-based building blocks may also be prepared by chemoenzymatic methods (Martin, B. D. et. al. Macromolecules, 1992, 25, 7081), for example in which Pseudomonas cepacia catalyzes the transesterification of monosaccharides with vinyl acrylate in pyridine or by the direct addition of an acrylate (Piletsky, S., Andersson, H., Nicholls, Macromolecules, 1999, 32, 633-636). Other functional groups may be present, as numerous derivatized carbohydrates are known to those familiar with the art of carbohydrate chemistry.

Besides carbohydrate-based building blocks, other examples of acrylate- or acrylamide-derivatized polymeric building blocks include polyethylene glycol-based molecules, such as polyethyleneglycol diacrylate.

Polyamide Formation: Oligomeric cationic building blocks such as chitosan or polylysine, may be crosslinked with multifunctional anions such as poly(aspartic acid) to form nanoparticles for use in the present invention.

Chemoselective Polymerizations: Chemoselective building blocks may also be used to form the nanoparticle. A representative example of this strategy may be the use of a polysaccharide that has been partially oxidized to contain numerous aldehydes within a reverse microemulsion. A di(amino-oxy) containing compound, such as that made from ethylene diamine (Reaction Scheme 8) can then be used as a crosslinking agent, with aldehydes of the oxidized sugar reacting with the amino-oxy functionalities.

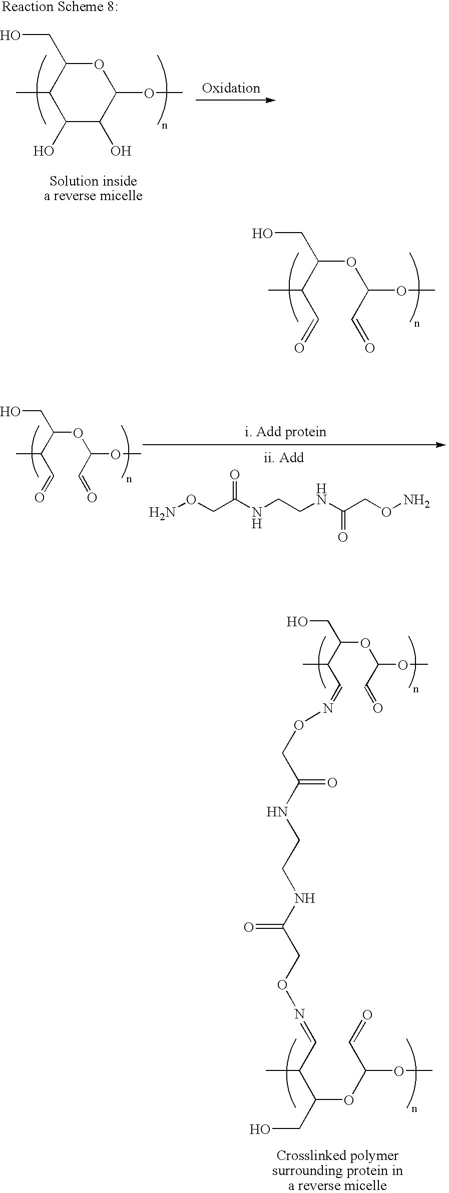

Nanoparticle Core Fabrication in Reverse Microemulsions: Where the nanoparticle is comprised of hydrophilic building blocks, the PFNs are fabricated by first forming nanoparticle cores through the crosslinking of hydrophilic monomeric building blocks solubilized in the dispersed water phase of a reverse microemulsion. Reverse microemulsions for nanoparticle core fabrication are formed by combining aqueous buffer or water, building blocks, organic solvent, surfactants and initiators in the appropriate ratios to yield a stable phase of surfactant-stabilized aqueous nanodroplets dispersed in a continuous oil phase. Stable reverse microemulsion formulations can be found using known methods by those skilled in the art. They are discussed, for example, in Microemulsion Systems, edited by H. L. Rosano and M. Clausse, New York, N.Y.: M. Dekker, 1987, and in Handbook of Microemulsion Science and Technology, edited by P. Kumar and K. L. Mittel, New York, N.Y.: M. Dekker, 1999. In this invention, an aqueous phase with solubilized hydrophilic building blocks is added to an organic solvent containing one or more solubilized surfactants to form a reverse microemulsion.

The dispersed aqueous phase contains hydrophilic building blocks solubilized at about 5 to about 65 wt %, preferably about 5 to about 25 wt %, most preferably 5 to 15 wt %. In the most preferred composition, after polymerization the resulting nanoparticles are more hydrophilic and soluble compared to higher polymer-content nanoparticles, and the amount of breakdown products is small relative to higher polymer-content nanoparticles. While not wishing to be bound by theory, the use of high water-content cores also may reduce immunogenicity in end uses, because there is less foreign surface for immune system components to recognize. The high water content also provides compliancy through a more flexible polymer network. Thus, when attaching to cell surface receptors, the nanoparticles are able to conform to the cell surface, allowing more surface receptors to be bound. Binding more receptors may allow the nanoparticle to better function as an antagonist. Additionally, while not wishing to be bound by theory, it is believed that PFN cell surface coverage can inhibit other cell signaling pathways.

Polymerization of the building blocks in the nanodroplets of the dispersed aqueous phase of the reverse microemulsion follows standard procedures well-known to those skilled in the art (see, for example, Odian G. G.; Principles of Polymerization, 3^{rd} Ed., Wiley, New York, 1991; L. H. Sperling, Introduction to Physical Polymer Science, Chapter 1, pp. 1-21, John Wiley and Sons, New York, 1986; and R. B. Seymour and C. E. Carraher, Polymer Chemistry, Chapters 7-11, pp. 193-356, Dekker, New York, 1981). Preferably, the building blocks possess functional/crosslinkable groups, such as acrylates and acrylamides, amenable to free-radical polymerization and polymerization can be induced through the combination of UV initiators and UV light, redox-coupled free-radical initiators or thermal initiators and heat. The organic solvent and non-reactive surfactants are removed after polymerization to yield crosslinked, water-soluble nanoparticles.

The size of the nanodroplets of the dispersed aqueous phase is determined by the relative amounts of water, surfactant and oil phases employed. Surfactants are utilized to stabilize the reverse microemulsion. These surfactants do not include crosslinkable moieties; they are not building blocks. Surfactants that may be used include commercially available surfactants such as Aerosol OT (AOT), polyethyleneoxy(n)nonylphenol (Igepal™, Rhodia Inc. Surfactants and Specialties, Cranbrook, N.J.), sorbitan esters including sorbitan monooleate (Span® 80), sorbitan monolaurate (Span® 20), sorbitan monopalmitate (Span® 40), sorbitan monostearate (Span® 60), sorbitan trioleate (Span® 85), and sorbitan tristearate (Span® 65), which are available, for example, from Sigma (St Louis, Mo.). Sorbitan sesquioleate (Span® 83) is available from Aldrich Chemical Co., Inc. (Milwaukee, Wis.). Other surfactants that may be used include polyoxyethylenesorbitan (Tween®) compounds. Exemplary cosurfactants include polyoxyethylenesorbitan monolaurate (Tween® 20 and Tween® 21), polyoxyethylenesorbitan monooleate (Tween® 80 and Tween® 80R), polyoxyethylenesorbitan monopalmitate (Tween® 40), polyoxyethylenesorbitan monostearate (Tween® 60 and Tween® 61), polyoxyethylenesorbitan trioleate (Tween® 85), and polyoxyethylenesorbitan tristearate (Tween® 65), which are available, for example, from Sigma (St Louis, Mo.). Other exemplary commercially available surfactants include polyethyleneoxy(40)-sorbitol hexaoleate ester (Atlas G-1086, ICI Specialties, Wilmington Del.), hexadecyltrimethylammonium bromide (CTAB, Aldrich), and linear alkylbenzene sulfonates (LAS, Ashland Chemical Co., Columbus, Ohio).

Other exemplary surfactants include fatty acid soaps, alkyl phosphates and dialkylphosphates, alkyl sulfates, alkyl sulfonates, primary amine salts, secondary amine salts, tertiary amine salts, quaternary amine salts, n-alkyl xanthates, n-alkyl ethoxylated sulfates, dialkyl sulfosuccinate salts, n-alkyl dimethyl betaines, n-alkyl phenyl polyoxyethylene ethers, n-alkyl polyoxyethylene ethers, sorbitan esters, polyethyleneoxy sorbitan esters, sorbitol esters and polyethyleneoxy sorbitol esters.

Other surfactants include lipids, such as phospholipids, glycolipids, cholesterol and cholesterol derivatives. Exemplary lipids include fatty acids or molecules comprising fatty acids, wherein the fatty acids include, for example, palmitate, oleate, laurate, myristate, stearate, arachidate, behenate, lignocerate, palmitoleate, linoleate, linolenate, and arachidonate, and salts thereof such as sodium salts. The fatty acids may be modified, for example, by conversion of the acid functionality to a sulfonate by a coupling reaction to a small molecule containing that moiety, or by other functional group conversions known to those skilled in the art.

Additionally, polyvinyl alcohol (PVA), polyvinylpirolidone (PVP), starch and their derivatives may find use as surfactants in the present invention.

Cationic lipids may be used as cosurfactants, such as cetyl trimethylammonium bromide/chloride (CTAB/CTAC), dioctadecyl dimethyl ammonium bromide/chloride (DODAB/DODAC), 1,2-diacyl-3-trimethylammonium propane (DOTAP), 1,2-diacyl-3-dimethyl ammonium propane (DODAP), [2,3-bis(oleoyl)propyl]trimethyl ammonium chloride (DOTMA), and [N-(n′, N′-dimethylaminoethane)-carbamoyl]cholesterol, dioleoyl) (DC-Chol). Alcohols may also be used as cosurfactants, such as propanol, butanol, pentanol, hexanol, hepatnol and octanol. Other alcohols with longer carbon chains may also be used.

Nanoparticle Core Functionalization: After the assembled building blocks are crosslinked to form the nanoparticle, the nanoparticle surface is functionalized with Type 2 high affinity peptides. The HAPs can be linked either directly or through a linker molecule to the surface of the nanoparticle, as discussed previously herein with respect to the block copolymer nanoparticles. In a linker configuration, part or all of the high affinity peptides are “displayed” at the end terminus of the tether. Therefore, in one application of the invention, the PFN consists of a high affinity peptide displayed at the surface of a biodegradable nanoparticle core as described previously herein.

In another embodiment of the patent, the PFN consists of a high affinity peptide linked to the surface of the previously described nanoparticle core via a linker molecule, the linker comprising preferentially PEG.

For each of these embodiments, it is possible to functionalize the nanoparticles with several coupling strategies, varying both the order of addition of the different components and the reactive chemical moieties used for the coupling.

Concerning the sequence in which the components are attached to one another, for example a PFN consisting of a high affinity peptide linked to a hydrophilic polymeric nanoparticle core by a linker molecule containing PEG can be synthesized in the following manners: The nanoparticle core is first reacted with a di-functional PEG-containing tether, followed by functionalization of the free terminus of a portion of the PEG chain with a high affinity peptide. Alternatively, the high affinity peptide is coupled first to the PEG-containing tether, followed by the attachment of the other PEG terminus to the nanoparticle core. Finally, in one embodiment of the invention, the PEG-containing tether linked to the high affinity peptide directly originates from the initial step-by-step solid-supported synthesis of the polyamino acid.

Concerning the coupling method(s), several combinations of reactive moieties can be chosen to graft the high affinity peptide to the nanoparticle core and to react the tether with the nanoparticle. In using a series of orthogonal reaction sets, varying some of the core building blocks and/or tethering arms, it is also possible to graft different peptides onto the same nanoparticle core and/or different enhancers onto the nanoparticle core in well-controlled proportions. Reactions using orthogonal reactive pairs can be done simultaneously or sequentially.

As far as reaction conditions are concerned, it is preferable to functionalize the nanoparticle in an aqueous system. The surfactants and the oil phase, residual from the synthesis of the nanoparticle core, can be removed through the use (singularly or in combination) of solvent washing, for instance using ethanol to solubilize the surfactant and oil while precipitating the polymer nanoparticles; surfactant-adsorbing beads; dialysis; or the use of aqueous systems such as 4M urea. Methods for surfactant removal are known in the art.

The high affinity peptide must contain a functionality that allows its attachment to the nanoparticle surface. Preferentially, although not necessarily, this functionality is one member of a pair of chemoselective reagents selected to aid the coupling reaction. (Lemieux, G., Bertozzi, C., Trends in Biotechnology, 1998, 16, 506-513). For example, when the nanoparticle surface (and/or linkers grafted to its surface) displays an α-carbonyl functionality, the peptide may be attached through a sulfhydryl moiety. A sulfhydryl moiety in the peptide structure can be accomplished through inclusion of a cysteine residue.

Coupling is also possible between a primary amine on the nanoparticle or the linker terminus and a carboxylic acid on the peptide. A carboxylate in the peptide structure can be found either on its terminal amino acid, for linear peptides, or through the inclusion of aspartic or glutamic acid. The opposite configuration, where the carboxylic acid is on the nanoparticle and a primary amine belongs to the peptide, is also easily accessible. Many polymerizable building blocks contain acidic moieties, which are accessible at the surface of the beads after their polymerization. As for the high affinity peptide, a primary amine function can be found either at its N-terminus (if it is linear) and/or via introduction of a lysine residue.

Another example of reactive chemical pairs consists of the coupling of a sulfhydryl with a maleimide moiety. The maleimide function can be easily introduced, either on a peptide, a linker, or the surface of the nanoparticles, by reacting other common functionalities (such as carboxylic acids, amines, thiols or alcohols) with (preferentially commercially available) short linkers, with methods known to one of skill in the art, such as described for example by G. T. Hermanson in Bioconjugate Techniques, Academic Press Ed., 1996. High affinity peptides can also be coupled to the nanoparticle and/or the tether with a reaction between an amino-oxy function and an aldehyde or ketone moiety. The amino-oxy moiety (either on the nanoparticles or in the peptide) can be introduced, starting from other common functionalities (such as amines for example), by a series of transformations known to those skilled in the art. In the same way, aldehyde- or ketone-containing particles and aldehyde-containing peptides are readily synthesized by known methods.

The resulting peptide-functionalized nanoparticles may be used immediately, may be stored as a liquid solution, or may be lyophilized for long-term storage.

III. Use of Enhancer Molecules

Optionally, the nanoparticle surface can also be functionalized by molecules other than peptides. Such molecules, which are referred to herein as “enhancers”, preferentially consist of small molecules belonging to one or more of the following categories: molecules enhancing uptake by certain cells (for example, folate used to facilitate uptake by certain cancer cells), molecules providing recognition for specific targeting (such as short-chain polysaccharides), molecules allowing the tracking of the PFN in the animal or plant tissue in which they accumulate (for example dyes, fluorescent or radiolabeled molecules), or any molecule other than a high affinity peptide providing a beneficial therapeutic effect. Similarly to the high affinity peptides, these enhancers can be attached directly to the surface of the PFN or displayed at the end of a linker molecule.

Generally, attachment of polymer core, PEG, enhancer, and HAP components may be accomplished through standard bioconjugation methods. For example, for coupling the carboxy terminus of the peptide to the amine terminus of the core or vice versa, a carbodiimide such as EDC may be used. For coupling amino groups on the core to amino groups on the peptide, glutaraldehyde may be used. These examples are not restrictive; those skilled in the art can choose from multiple coupling reagents and methodologies (see for instance Jones, J., Amino Acid and Peptide Synthesis, Oxford University Press, New York 1992; Bodanszky, M; Bodanszky, A., The Practice of Peptide Synthesis, Springer-Verlag, New York 1994; G. T. Hermanson, Bioconjugate Techniques, Academic Press, San Diego, 1996). Alternatively, complimentary pairs of chemoselective coupling reagents may be employed, with one member of the pair incorporated into the PAA and the other member of the pair incorporated into the HAP. Indeed, this strategy has been used with success in the preparation of large polypeptide sequences. (Lemieux, ibid.). Also, new advances in the preparation of end-functionalized polypeptides (Curtin, S. A., Deming, T. J., J. Am. Chem. Soc., 1999, 121, 7427-7428) may allow for the direct synthesis of the HAP terminal copolymer structure, avoiding the necessity of the coupling chemistry outlined above.

IV. Protein-Controlled Release

In certain embodiments, nanoparticles of the present invention are useful for the controlled release of therapeutic peptides or proteins. The nanoparticles of the present invention may be comprised of proteins or protein fragments that act on the nervous system, endocrine system, cardiovascular system, blood circulatory system, respiratory system, reticuloendothelial system, skeletal system, skeletal muscles, smooth muscles, immunological system, reproductive system, and cancerous tissues, among others.

Proteins that may be advantageously employed in the present invention include, but are not limited to, those naturally produced by the human body, recombinant versions of such proteins, and derivatives and analogs of such proteins, and soluble portions of receptors. For example, cytokines such as interleukins and interferons, and growth factors and soluble fractions of cytokine and growth factor receptors may be used. Cytokines are often low molecular weight glycoproteins. Because glycoproteins are generally hydrophilic in nature, they are particularly amenable for use in the present invention. Hydrophilic proteins are expected to be incorporated to an unsatisfactory extent in block copolymer nanoparticles presently known in the art, because it is expected to be thermodynamically preferable for the hydrophilic proteins to remain in solution outside of the nanoparticies. However, in the present invention the high affinity peptides serve to bind the hydrophilic protein to a block copolymer, and subsequently to or within a nanoparticle.

Thereapeutic-Containing Particles Formed From PAA Blocks: Block copolymers, at least some of which are Type 1 peptide-functionalized block copolymers, are added to an aqueous solution. DMF may be employed to facilitate solubilization. Second, the therapeutic protein is solubilized in an aqueous solution. The Type 1 peptide may noncovalently bind to the therapeutic protein before, during or after formation of the nanoparticles in solution. The block copolymers then spontaneously coalesce into nanoparticles with removal of DMF. The end result is the formation of nanoparticles comprised in part of proteins or protein fragments of therapeutic value, which are at least partially enclosed or surrounded by the nanoparticle.

The block copolymer-peptide functionalized with HAPs and complexed with the therapeutic protein has a different structure and will behave differently than the block copolymers that are not functionalized with high affinity peptides. For instance, with a relatively hydrophilic protein, the block copolymer-protein complex will be larger and more hydrophilic than the block copolymer alone. Thus, it may be advantageous to reduce the size of or eliminate entirely the hydrophilic block for block copolymers functionalized with Type 1 peptides.

High affinity peptides may be attached to the PAA by a variety of methods familiar to those skilled in the arts of peptide synthesis or organic chemistry. For example, the carboxylic acid terminus of the HAP may be coupled to the amine terminus of the PAA through the use of activated esters or carbodiimide coupling chemistry frequently used in peptide synthesis. Alternatively, complimentary pairs of chemoselective coupling reagents may be employed, with one member of the pair incorporated into the PAA and the other member of the pair incorporated into the HAP. Indeed, this strategy has been used with success in the preparation of large polypeptide sequences. (Lemieux, G., Bertozzi, C., Trends in Biotechnology, 1998, 16, 506-513 and the references cited therein.) It may also be envisioned that the method of Deming et al. may be employed to grow a PAA block off of a HAP (Curtin, S. A., Deming, T. J., J. Am. Chem. Soc., 1999, 121, 7427-7428) (Reaction Scheme 9). In this method, a HAP is appropriately functionalized (protection of reactive functional groups of the HAP may be necessary prevent subsequent inhibition of the NCA polymerization) to act as an initiating ligand for the polymerization of an NCA, yielding the HAP-PAA material. If the HAP or PAA was protected used in a protected state, deprotection of the HAP-NCA material may be necessary in order to generate the active species.

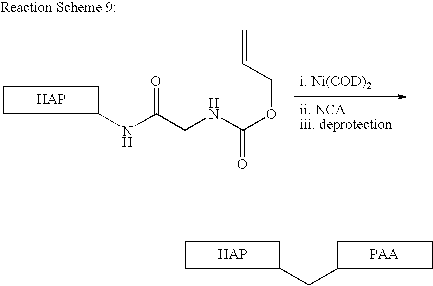

Retention of the therapeutic protein within the nanoparticle may be controlled through peptide affinity and peptide concentration. An optimum in peptide affinity/peptide concentration is likely. If the nanoparticle binds the protein too weakly, the protein will be released too quickly. If the binding is too strong, the protein may be sequestered in the nanoparticle for longer than is desired.

Therapeutic-Containing Particles Formed From Polycaprolactone Blocks: In a similar fashion, it may be desirable to attach the HAP to polycaprolactone. This may be accomplished in a number of ways to those skilled in the arts of peptide synthesis or organic chemistry. For example, the terminal hydroxyl of the polycaprolactone may be converted into a leaving group, such as that that would result from the reaction of the alcohol with p-toluenesulfonyl chloride. The resulting sulfonic ester could be displaced by the amine of HAP, yielding a covalent bond between the HAP and the polycaprolactone (Reaction Scheme 10).

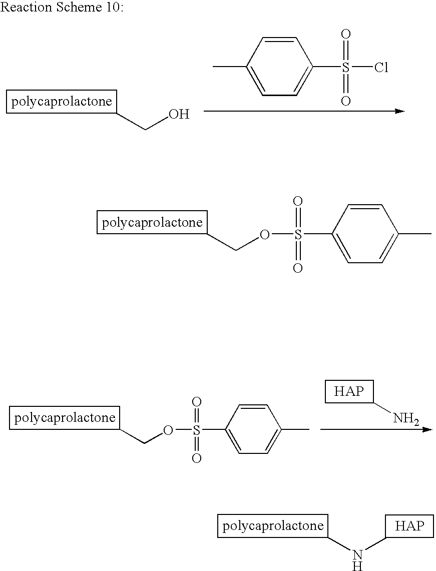

Alternatively, a polycaprolactone block may be grown off of a suitable protected and functionalized HAP through the use of aluminum porphyrins. This example would have a similar scheme as that displayed in Scheme 10, except that a HAP would take the place of the polysaccharide demonstrated in that picture. After the protective groups have been removed, the active HAP-polycaprolactone material could be isolated.

Therapeutic-Containing Particles Formed In Reverse Microemulsions: Proteins may also be sequestered in the core of a nanoparticle formed using the reverse microemulsion method. For this embodiment, it is important that the nanoparticle be formed around the active therapeutic protein without attenuting the protein's activity. This may be accomplished by the use of the chemoselective reagents in the formation of the nanoparticle in the dispersed aqueous phase of a reverse microemulsion as discussed previously in this disclosure. A representative example of this strategy is the use of a polysacharide that has been partially oxidized to contain numerous aldehydes within a reverse microemulsion. A high affinity peptide with a ketone functionality is also included in the solution. The ketone containing peptide can be formed by the functionalization of the amine terminus (or lysine residue side chains) with levulinic acid. A therapeutic protein to which the HAP binds to is also included in the microemulsion. A di(amino-oxy) containing compound, such as that made from ethylene diamine (Reaction Scheme 11) is then used to both attach the HAP to the polysaccharide and to crosslink the polysaccharide cores. Thus, a crosslinked network surrounding the therapeutic protein is formed.

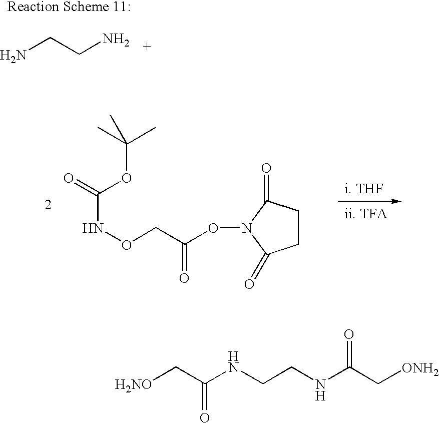

The use of the HAP serves to increase the retention of the protein in the nanoparticle core. Retention of the therapeutic protein within the nanoparticle is thus controlled through peptide affinity, peptide concentration, and network crosslink density.

V. Targeted Delivery of Therapeutic Proteins

Another embodiment of the invention provides a method for the controlled delivery of therapeutic proteins to the vicinity of the targeted cell or tissue type. The nanoparticles of this embodiment are comprised of four types of molecular structures: nanoparticle matrix molecules, Type 1 peptides with high affinity to therapeutic proteins, Type 2 peptides with high affinity to proteins expressed on certain cells or in certain tissues, and therapeutic proteins. Thus, when administered as a therapeutic, the nanoparticles containing the therapeutic protein will concentrate in tissue or on cell surfaces expressing a targeted protein. The therapeutic protein-peptide affinity also allows the retention of the therapeutic protein in the nanoparticle in use, extending circulation time of the protein. One or more peptides capable of high affinity binding to the therapeutic protein may be used.

The proteins can be included both in nanoparticles fabricated from block copolymer structures and nanoparticles formed in the dispersed phase of reverse microemulsions as disclosed previously herein. Nanoparticle surface functionalization with Type 2 peptides for both block copolymer nanoparticle structures and nanoparticles formed in the dispersed phase of reverse microemulsions has also been delineated in this disclosure.

VI. PFN Attributes

As practiced in the invention, the water-soluble peptide-functionalized nanoparticles (PFNs) have several attributes that make them excellent therapeutic candidates. Because of their small size, the nanoparticles can be used in vivo (in a mammalian body, e.g.) as bioactive entities. In in vivo applications, when compared to peptides alone, the PFNs disclosed herein may advantageously possess increased affinity and longer circulation time. The nanoparticles will be metabolized to nontoxic substances in the body.

The nanoparticles of the present invention have a high water content. By “high water content” is meant that, with respect to the PFNs comprising block copolymers, the core may be from 85 wt % to greater than 95 wt % water, with the remainder being polymeric material. Those PFNs comprising hydrophilic polymers crosslinked in the dispersed aqueous phase of reverse microemulsions have a core of about 35 to about 95 wt % water, preferably about 75 to about 95 wt % water, and most preferably 85 to 95 wt % water. Thus, the amount of breakdown products is less than particles with a higher polymer concentration. The high water content cores also can reduce immunogenicity, because there is less surface for immune system components to interact with. The high water content also provides compliancy. Thus, when attaching to cell surface receptors, the nanoparticles are able to conform to the cell surface, allowing more surface receptors to be bound. Binding more receptors allows the nanoparticle to better function as an antagonist. Additionally, while not wishing to be bound by theory, it is believed that PFN cell surface coverage can inhibit other cell signaling pathways.

Enhanced therapeutic performance may result from the multivalent structure of the PFNs. By “multivalent” is meant that there are two or more high affinity recognition elements per nanoparticle. Multivalency allows the nanoparticles to bind at multiple points simultaneously. Multivalency also provides a high local concentration of peptides in the vicinity of a target molecule. While not wishing to be bound by theory, it is believed that this high local concentration can increase the percentage of bound targets.

Furthermore, the PFN fabrication strategy outlined herein allows the fabrication of numerous fine-tuned heterofunctionalized entities whose surface composition can be tailored according to the desired properties. It is possible, by varying some of the core building blocks and/or tethering arms, to graft different peptides and/or enhancer molecules (for example PEG chains, delivery enablers such as folates, or small therapeutic molecules) to nanoparticles in well-controlled proportions by utilizing a series of orthogonal reaction sets (amine-acid, aminooxy-ketone and bromoacetamide-sulfhydryl). The use of PEG as an enhancer may, for example, provide steric hindrance to prevent digestive enzymes from cleaving off the attached peptide; or provide a hydration layer near the surface of the PFN; or minimize immunogenic responses of the PFNs, making them stealthy.

An additional advantage of the present application is that more than one type of peptide molecule may be incorporated into a single nanoparticle. This can allow, for instance, therapeutic peptides to be targeted to a desired cell type, tissue, or organ using a targeting peptide.

Another advantage of the nanoparticles disclosed in this invention is their utility as cancer therapeutics. The leaky vasculature found in tumors allows these nanoparticles to leave the blood stream and concentrate in tumors. This effect, described as enhanced permeability and retention (EPR) for macromolecular agents, has been observed to be universal in solid tumors (H. Maeda, et.al., J. Controlled Release, 2000, V65, p.271-284). The key mechanism for the EPR effect for macromolecules is retention, whereas low-molecular-weight substances are not retained but are returned to circulating blood by diffusion. PFNs of diameters from 5 to 100 nm can thus accumulate in solid tumors. Thus, the nanoparticles of the present invention will naturally concentrate in tumors even prior to target binding, providing for greater efficacy and less systemic toxicity.

The nanoparticles and the breakdown products of this invention are designed to be non-toxic and eliminated from the body. They may have degradable, preferably carbohydrate-based, PAA-based, or PEG-based cores, with the rate of degradation controlled by the identity of the sugar, crosslink density, and other features. Thus, the nanoparticles can be metabolized in the body, preventing undesirable accumulation in the body. The nanoparticles can be administered by injection (subcutaneous, intravenous, intramuscular, intradermal, intraperitoneal, intracerebral, or parenteral). The nanoparticles may also be suitable for nasal, pulmonary, vaginal, ocular delivery and oral ingestion. The nanoparticles may be suspended in a pharmaceutically acceptable carrier for administration.

The nanoparticles of this invention are also useful in separation and diagnostic applications in an environment containing a targeted cell or tissue type because of their biomolecular recognition capabilities.

VIII. Therapeutic Targets and Therapeutic Applications

One area in particular in which the nanoparticles of the present invention may be advantageously used is the treatment of cancer. Tumors are known to have leaky vasculature that allows proteins and larger entities such as nanoparticles to concentrate within the tumor. Nanoparticles of the present invention that employ such proteins and protein fragments may find use as anticancer therapeutics. For example, a soluble portion of epidermal growth factor receptor (EFGr) and a soluble portion of vascular endothelial growth factor receptor (VEGFr) may be employed. The release of the soluble portion of receptors such as EGFr and VEGFr within a tumor may serve to bind to the growth factors EGF and VEGF before EGF and VEGF can bind to their receptors on the cell surface. Such use can serve to slow the growth of tumors and slow angiogenesis within the tumor.

In another example application of the technology, nanoparticles that deliver VEGF-2 to cardiac tissue may be used to stimulate the growth of new cardiac arteries, and thus may act as a therapeutic in the treatment of heart disease.

Additional active agents that may be employed in the present invention include interleukins such as IL-2 and colony stimulating factors such as GM-CSF, both of which purportedly stimulate the immune system, and tumor necrosis factors.

Reagents and starting materials in some embodiments can be obtained commercially from chemical distributors such as Sigma-Aldrich (St Louise, Mo. and Milwaukee, Wis.), Kodak (Rochester, N.Y.), Fisher (Pittsburgh, Pa.), Pierce Chemical Company (Rockford, Ill.) and Carbomer Inc. (Westborough, Mass.). Radcure (Smyrna, Ga.), and Polysciences (Niles, Ill.). PEG molecules may be purchased through Shearwater Polymers (Huntsville, Ala.). Additionally, chemical product directories and resources such as and <http://pubs.acs.org/chemcy/> may be used to locate starting materials.

Peptides to be used as high affinity binders can be purchased from many sources, one being Peptide Biosynthesis (www.peptidebiosynthesis.com).

### EXAMPLES

### Example 1

Poly(ethylene oxide-b-ε-caprolactone) (Mw=1.5×10⁴, poly(ethylene oxide) block between 15 and 25% of the total weight), is prepared by the method of Gan, Z.; Jiang, B.; Zhang, J. J. Appl. Polym. Sci. 1996, 59, 961. After isolation, the product is dissolved in THF and added to an aqueous solution of NaSH. Once the reaction with NaSH is complete, the solution is acidified to a neutral pH in a fume hood by the addition of HCl and all volatile compounds are removed under vacuum, leaving a water solution of only slightly reduced volume. The product is dissolved in THF again, under a protective blanket of nitrogen, and centrifuged to remove salts. The volume of THF is reduced under vacuum, and the resulting solution is added dropwise to a rapidly stirring beaker of degassed water, containing 10 to 1000 times more water than THF being added to it. The resulting solution is placed under a vacuum to remove THF. A modified version of Arg-Gly-Asp-D Phe-Lys (which contains the bromoacetamide functionality on the terminal lysine) is then added, dissolved in a minimal amount of DMF to assist in dispersion of the peptide in the water phase, to the aqueous solution. The nanoparticle solution is concentrated under vacuum and dialyzed against a large reservoir of water to remove unreacted peptide and salt side products. Lyophilization of the dialyzed solution, after addition of a carbohydrate cryoprotector, yields peptide-functionalized poly(ethylene oxide-b-ε-caprolactone) nanoparticles.

In an alternative formulation, 1-5% of the ε-caprolactone block may be substituted by 1,2-epoxy-4-pentanone ethylene ketal. After synthesis of the polymer and addition of a thiol to the end, the cyclic ketals are removed by exposing the polymer to a mildly acidic aqueous solution. The micelles are then made and functionalized with the peptide as outlined above. After addition of the peptide, a crosslinking agent such as diamino-oxy functionalized PEG (Mw=5000) is added to the water solution in order to crosslink the interior of the nanoparticle. The particle is then purified and isolated as before.

### Example 2

Following the procedure of Haddleton (in Haddleton, D. M.; Ohno, K., Biomacromolecules, 2000, 1, 152), β-cyclodextran is ring opened and modified to yield a protected polysaccharide with only the terminal anomeric hydroxyl remaining unprotected. This compound, after drying under vacuum at a slightly elevated temperature, is reacted with (5,10,15,20-tetraphenylporphinato)aluminum ethyl, yielding the active polymerization initiator species. This species is used to polymerize ε-caprolactone via the method of Endo (in Endo, M.; Aida, T.; Inoue, S. Macromolecules, 1987, 20, 2982). After isolation and hydrolysis of the protecting groups on the polysaccharide, the polymer is reacted with 1,1′-carbonyldiimidazole in DMF for three days. Then, ethylene diamine is added and the reaction is allowed to progress for two more days. This product is then reacted with bromoacetic acid anhydride. The product is isolated, dissolved in DMF, and added dropwise to an aqueous solution. After micelle formation, the peptide Arg-Gly-Asp-D Phe-Lys-Cys is added to the aqueous solution as a solution in DMF. The nanoparticle solution is concentrated under vacuum and dialyzed against a large reservoir of water to remove unreacted peptide and salt side products. Lyophilization of the dialyzed solution, after addition of a carbohydrate cryoprotector, yields peptide-functionalized nanoparticles.

In an alternative formulation, 1-5% of the ε-caprolactone block may be substituted by 1,2-epoxy-4-pentanone ethylene ketal. After synthesis of the polymer, the cyclic ketals are removed by exposing the polymer to a mildly acidic aqueous solution and the polysaccharide is modified as described above. The micelles are then made and functionalized with the peptide as outlined above. After addition of the peptide, a crosslinking agent such as diamino-oxy functionalized PEG (Mw=5000) is added to the water solution in order to crosslink the interior of the nanoparticle. The particle is then purified and isolated as before.

### Example 3

Difunctional PEG (Mw=5000, bromoacetamide-functionalized) is reacted with a poly(γ-benzyl glutamate₁₁-co-cysteine₁) block copolymer synthesized by the method of Deming (Deming, T. J., Nature 1997, 390, 386-389; Deming, T. J., J. Am. Chem. Soc. 1997, 119, 2759-2760). After isolation, the polymer is dissolved in DMF and added dropwise into an aqueous solution. After micelle formation, the peptide Arg-Gly-Asp-D Phe-Lys-Cys, dissolved in a minimal amount of DMF to assist in dispersion of the peptide in the water phase, is then added to the aqueous solution. The nanoparticle solution is concentrated under vacuum, and dialyzed against a large reservoir of water to remove unreacted peptide and salt side products. Lyophilization of the dialyzed solution, after addition of a carbohydrate cryoprotector, yields peptide-functionalized nanoparticles.

In an alternative formulation, 10-20% of the γ-benzyl glutamate of the poly(γ-benzyl glutamate₁₁-co-cysteine₁) block may be substituted by lysine. After synthesis of the polymer, the lysine residues are selectively deprotected in the presence of the γ-benzyl glutamate. The micelles are then made and functionalized with the peptide as outlined above. After addition of the peptide, 1,3-butadiene diepoxide, dissolved in DMF, is added to the water solution to crosslink the nanoparticle interior. The particle is then purified and isolated as before.

### Example 4

Using the method of Deming (Deming, T. J., Nature 1997, 390, 386-389; Deming, T. J., J. Am. Chem. Soc. 1997, 119, 2759-2760) a block co-polymer of poly(γ-benzyl-L-glutamate₈®-co-leucine₂₀) is prepared from the polymerization of N-carboxyanhydrides. After deprotection of the glutamate block with HBr/acetic acid, the block copolymer is dissolved in DMF, which is added slowly to an aqueous solution to induce nanoparticle formation. The amine terminus of the glutamate block is coupled to the carboxylate terminus of the peptide Arg-Gly-Asp-D Phe-Lys (in which the side chain amine is protected by a photo-labile protecting group) by action of 1-(3-dimethylaminopropyl)-3-ethylcarbodiimide hydrochloride. Once the coupling reaction is complete, the peptide is deprotected, and the reaction mixture is dialyzed against a large reservoir of water to remove unreacted peptide and salt side products. Lyophilization of the dialyzed solution, after addition of a carbohydrate cryoprotector, yields peptide-functionalized nanoparticles.

In an alternative formulation, 10-20% of the leucine of the poly(γ-benzyl-L-glutamate₈₀-co-leucine₂₀) block may be substituted by lysine. After synthesis of the polymer, the lysine and glutamate residues are deprotected with HBr/acetic acid and trifluoroacetic acid. The micelles are then made and functionalized with the peptide as outlined above with the exception that the photo-labile group on the peptide is removed after crosslinking (described in the next step). After addition of the peptide, 1,3-butadiene diepoxide, dissolved in DMF, is added to the water solution to crosslink the nanoparticle interior, and the photo-labile group is removed. The particle is then purified and isolated as before.

### Example 5

PAA block copolymer chains with an AB structure comprised of a hydrophobic block of a random copolymer of Leu and Val (Leu-Val) with a molecular weight between 1000 to 10,000 Da, and a hydrophilic block comprised of Glu with a molecular weight of between 5,000 to 20,000, thus with a total molecular weight of approximately 6,000 and 30,000 Da, are formed through a controlled polymerzation mechanism (following the procedure of Deming, supra, e.g.). The acid groups of the Glu residues are kept methylated and the carboxy terminus is protected to aid in attachment of the high affinity peptide in the next step.

A peptide comprised of Leu-Ser-Trp-His-Pro-Gly is purchased. Peptides containing this sequence have been shown to bind to streptavidin with a binding constant in the 10⁻⁴ M range. The carboxyl terminus of this peptide is attached to the amine terminus of the PAA chain through EDC coupling (G. T. Hermanson, Bioconjugate Techniques, Academic Press, San Diego, 1996) to produce a PAA-HAP.

To form the nanoparticles, both PAA and PAA-HAP chains are added to an aqueous solution. The weight ratio of PAA-HAP to PAA chains is between 1 to 1 and 1 to 100. Addition of between 1 to 50 mg of the polymer mixture to 1 mL of an aqueous solution that has a pH between 5 and 7 and containing between 10 and 500 mM NaCl results in spontaneous nanoparticles formation.

### Example 6

When the block copolymer is a PAA with the hydrophilic block comprised of acidic or basic residues, the pH of the reaction is chosen such that at least a portion of the hydrophilic ionizable monomers are ionized. For example, for assembly of Leu/Glu chains, the aqueous solution can range in pH from 3 to 13 and sodium chloride is included in the solution at a concentration between 0.01 and 0.5 M. The block copolymers then spontaneously coalesce into nanoparticles.

### Example 7

PAA block copolymer chains with an AB structure as in Example 5 or 6 are formed through a controlled polymerization mechanism (supra Deming). The acid groups of the Glu residues are kept methylated and the carboxy terminus is protected to aid in attachment of the high affinity peptide in the next step.

A peptide comprised of the Arg-Gly-Asp sequence is purchased. Peptides containing this sequence have been shown to bind to certain integrins with a binding constant in the nanomolar range. The carboxyl terminus of this peptide is attached to the amine terminus of the PAA chain through EDC coupling (G. T. Hermanson, Bioconjugate Techniques, Academic Press, San Diego, 1996) to produce a PAA-HAP.

To form the particles, both PAA and PAA-HAP chains are added to an aqueous solution. The weight ratio of PAA-HAP to PAA chains is between 1 to 1 and 1 to 100. Addition of between 1 to 50 mg of the polymer mixture to 1 mL of an aqueous solution that has a pH between 5 and 7 and containing between 10 and 500 mM NaCl results in spontaneous nanoparticles formation.

### Example 8

An aqueous phase is prepared comprised of 85 wt % buffer (10 mM PBS, 15 mM NaCl, pH 7.2), 8 wt % IMA (inulin multi-acrylamide), 6 wt % AM (acrylamide), and 1 wt % NOBA (sodium ornithine bromoacetamide acrylamide). An oil+surfactant phase is prepared by mixing Igepal CO-210, Igepal CO-720 and cyclohexane in a weight ratio of 1.0:1.3:9.0. Three grams (3 g) of aqueous phase are mixed with 30 g of the oil+surfactant phase, resulting in the formation of a reverse microemulsion. The reverse microemulsion contains surfactant-stabilized nanodroplets of aqueous phase dispersed in a continuous phase of cyclohexane. Photoinitiator(s) such as Irgacure and Darocur (Ciba) are then added at 0.01 part to. 1 part by weight photoinitiators to 100 parts by weight building blocks. The reverse microemulsion is then degassed, backfilled with N₂, and irradiated with a UV lamp for 20 min to 1 hour to polymerize the building blocks (IMA, AM, NOBA). Once polymerization is complete, the nanoparticles are precipitated by adding ethanol to the reverse microemulsion. Residual surfactants and solvents are removed by standard techniques such as dialysis and chromatography. At this point, the aqueous solution of nanoparticles may be lyophilized, leaving the nanoparticles as a flocculent solid that is readily dissolved in water. The nanoparticles can then be incorporated into an appropriate aqueous solution.

### Example 9

An aqueous phase is prepared comprised of 81 to 85 wt % buffer (10 mM PBS, 15 mM NaCl, pH 7.2), 8 wt % IMMA (inulin multi-methacrylate), 6 wt % AM (acrylamide), and 1 to 5 wt % DAA (diacetone acrylamide). An oil+surfactant phase is prepared by mixing Igepal CO-210, Igepal CO-720 and cyclohexane in a weight ratio of 1.0:1.3:9.0. Three grams (3 g) of aqueous phase are mixed with 30 g of the oil+surfactant phase, resulting in the formation of a reverse microemulsion. The reverse microemulsion contains surfactant-stabilized nano-droplets of aqueous phase dispersed in a continuous phase of cyclohexane. To the reverse microemulsion, 10 μL of a UV-photoinitiator stock solution (95 wt % toluene, 2.5 wt % Irgacure, and 2.5 wt % Darocur) is added. The reverse microemulsion is transferred to a 100 mL Schlenk tube. The contents of the tube are degassed using a water aspirator. The contents of the tube are aspirated for 5 minutes, followed by 1 minute of N₂ gas backfill into the tube. This aspiration/backfill step is repeated for a total of 3 cycles. The contents of the Schlenk tube are stirred and irradiated with a UV lamp for 1 hour to polymerize the building blocks (IMMA, AM, DAA). After polymerization is complete, the nanoparticles are precipitated by adding 9 mL of pure ethanol directly to the solution. The nanoparticle-containing pellet is resuspended in deionized water. Residual surfactants and solvents are removed by standard techniques (dialysis, chromatography, etc.). At this point, the aqueous solution of nanoparticles may be lyophilized.

### Example 10

An aqueous phase is prepared comprised of 81 to 85 wt % water, 14 wt % IMMA (inulin multi-methacrylate), and 1 to 5 wt % DAA (diacetone acrylamide). An oil+surfactant phase is prepared by mixing Igepal CO-210, Igepal CO-720 and cyclohexane in a weight ratio of 1.0:1.3:9.0. Three grams (3 g) of aqueous phase are mixed with 30 g of the oil+surfactant phase, resulting in the formation of a reverse microemulsion. The reverse microemulsion contains surfactant-stabilized nano-droplets of aqueous phase dispersed in a continuous phase of cyclohexane. To the reverse microemulsion is added an aqueous solution containing Eosin Y, where the photoinitiator represents from 0.001 to 0.1 wt % of the monomers mass. The reverse microemulsion is transferred to a 100 mL Schlenk tube, degassed with three cycles of freeze-thawing, followed by 1 minute of N₂ gas backfill into the tube. The contents of the Schlenk tube are stirred and irradiated with a visible light source of at least 100 W for 20 min to two hours to polymerize the building blocks. Once the polymerization is complete, the nanoparticles are precipitated by adding 9 mL of pure ethanol directly to the solution. The nanoparticle-containing pellet is resuspended in deionized water. Residual surfactants and solvents are removed by standard techniques (dialysis, chromatography, etc.). At this point, the aqueous solution of nanoparticles may be lyophilized.

### Example 11

A solution of polyethyleneimine (of 800 kDa molecular weight average; “PEI”) in a basic buffer (pH values ranging from 8 to 10) at 0.03 g/ml was added dropwise to a solution containing polyacrylic acid (1.8 kDA molecular weight average) at 6.5 mg/mL. After 15 minutes, the system has reached equilibrium. A ratio of 10 amino groups (from the PEI) per acid group (from the polyacrylic acid) gave stable narrowly monodisperse nanoparticles of 10 nm radius average.

### Example 12

An aqueous phase is prepared comprised of 85 wt % water and 15 wt % inulin. An oil+surfactant phase is prepared by mixing Igepal CO-210, Igepal CO-720 and cyclohexane in a weight ratio of 1.0:1.3:9.0. Three grams (3 g) of aqueous phase are mixed with 40 g of the oil+surfactant phase, resulting in the formation of a reverse microemulsion. One gram of a sodium periodate solution in water is added to the mixture, so that there is a maximum of one periodate equivalent per glucose monomer unit (from the inulin). The in-situ oxidation is allowed to proceed for ca 10 minutes before the protein to encapsulate (such as Interleukin-2) is added. A concentrated solution (of at least 1 g/mL) of bis[(2-amino-oxy)ethylamido]-(1,3-)propane in an aqueous methanol mixture (50/50 volume) is added to the microemulsion and allowed to react overnight (time not optimized). The nanoparticles obtained by the crosslinking of the bis[(2-amino-oxy)ethylamido]-(1,3-)propane with the aldehydes functions generated by the in-situ reduction of inulin contain entrapped interleukin-2. They are purified by precipitation in presence of ethanol and centrifugation and purified from the excess of unreacted reagents by dialysis.

### Example 13

Nanoparticles synthesized according to the preceding examples with an aqueous phase comprised of 85 wt % buffer (10 mM PBS, 15 mM NaCl, pH 7.2), 8 wt % IMMA (inulin multi-methacrylate), 6 wt % AM (acrylamide), from 0.3 to 3.0 wt % NOBA (Sodium ornithine bromoacetamide acrylamide) and from 0.3 to 3.0 wt % DAA (Diacetoneacrylamide) are dissolved in an aqueous solution. One typical procedure consists of dissolving the nanoparticles 1 to 5 g/L in a 0.05 M 2,2-bis(hydroxymethyl)-2,2′,2″-nitrilotriethanol buffer, pH 8, containing 1 to 10% vol methanol. The appropriate amount of thio- and N-hydroxy-aminofunctionalized polyethylene glycol(s) of average molecular weight, typically between 2,000 and 20,000, is added to the reaction mixture and the reaction is stirred overnight (reaction time not optimized). Peptide(s) can be grafted directly to the nanoparticles simultaneously with the PEG.

Different linkers can be used, depending on which functional moiety one would like to attach the peptide(s). As far as the two sets of reactions are orthogonal, it is possible to selectively control the relative quantities of two different peptides grafted on the surface. Ethylene-diamine bromoacetamide or 3-bromoacrylamidepropanal can be chosen as linker molecules. The linker molecules are added to the reaction mixture, ideally at least one equivalent to one equivalent of thiol-reactive functions present in the reaction media. If peptide is grafted after the FPLC column, a typical procedure consists of reacting the peptide at a 1 mg/mL concentration in the nanoparticles.

The grafting of peptide H₂N-WLWHPQFSSC-CO₂H onto the end of a dithiol-PEG chain via reaction of the cysteine residue's thiol with the bromoacetamide moiety leads to nanoparticles with a high binding capacity: Isothermal titration calorimetry gave an affinity binding of 3×10⁴ M for the free peptide toward Streptavidine, while the nanoparticles were found to have two types of binding sites, with one type having a binding constant of 1.6 (±0.9)×10⁻⁶ M, and a second type having a constant of 5.9 (±4)×10⁻⁷ M. The nanoparticle thus demonstrates a substantial improvement in binding compared to the unattached peptide.

### Example 14

Nanoparticles made by polymerization of 10 wt % IMMA (inulin multi-methacrylate), 4 wt % DM (diacetone acrylamide) and 1 wt % NOBA (sodium ornithine bromoacetamide acrylamide) are reacted with an excess of polyethylene glycol diamine of 6000 molecular weight average (H₂N-PEG₆₀₀₀-NH₂). The unreacted H₂N-PEG₆₀₀₀-NH₂ is eliminated by precipitation or any other method known to the art (such as SCC of FPLC). A solution of folic acid in a bicarbonate buffer (1 g/ml, pH 6.6) is added to the aminoPEG-functionalyzed nanoparticles suspended in water (10 mg nanoparticles/mL aqueous solution) and coupled with 1-(3-dimethylaminopropyl)-3-ethylcarbodiimide hydrochloride (EDC) and 1 equivalent N-hydroxysuccinimide. The nanoparticles can be purified by any techniques know of the art such as but not limited to dialysis, centrifugation or Size Exclusion Chromatography.

### Example 15

Following the method of Deming (Deming, T. J., Nature 1997, 390, 386-389; Deming, T. J., J. Am. Chem. Soc. 1997, 119, 2759-2760), poly(leucine) (Mw=2200) is prepared from the polymerization of leucine N-carboxyanhydride. In this case, the nickel polymerization catalyst is prepared with the HAP to erythropoietin serving as the initiating ligand following the method of Curtin (Curtin, S. A., Deming, T. J., J. Am. Chem. Soc., 1999, 121, 7427-7428). In this case, the amine terminus of the HAP to erythropoietin is protected with the allyloxycarbonyl (Alloc) protecting group, while the rest of the HAP to erythropoietin is protected with other protecting groups commonly used in peptide synthesis. After the polymer has been isolated, the HAP to erythropoietin is converted into the active form by deprotection of the side chains, and dissolved in DMF. The DMF solution is slowly added to an aqueous solution of erythropoietin. The resulting aqueous solution is dialyzed to remove DMF.

In an alternative formulation, 10-20% of the leucine of the poly(leucine) block may be substituted by lysine as a random copolymer. After synthesis of the polymer, the lysine residues are deprotected with trifluoroacetic acid. The lysine residues of the PAA block are then functionalized with levulinic acid, to include a ketone in the PAA block. The HAP to erythropoietin is then deprotected. Micelles are then formed as outlined above, in the presence of the erythropoietin, and di-amino-oxy functionalized PEG (Mw=5000), dissolved in DMF, is added to the water solution to crosslink the nanoparticle interior. The particle is then purified and isolated as before.

### Example 16

LEU/GLU block copolymer chains (PAA chains), with an AB structure and with each block approximately 10,000 to 20,000 Da and thus with a total molecular weight of 20,000 to 40,000 Da, are formed through a controlled polymerization mechanism (supra Deming). Also, LEU/GLU block copolymer chains (PAA chains), with an AB structure, with the LEU block approximately 10,000 to 20,000 Da and the GLU block between 1000 and 2000 Da and thus with a total molecular weight of 11,000 and 22,000 Da, are formed through a controlled polymerization mechanism (supra Deming). This second “short” chain structure is used for peptide coupling. The acid groups of the GLU residues are kept methylated and the carboxy terminus is protected to aid in attachment of the high affinity peptide in the next step.

A hexamer peptide that binds to EPO with an affinity constant in the micromolar to nanomolar range is purchased. The carboxyl terminus of this peptide is attached to the amine terminus of a “short” LEU/GLU PAA chain through EDC coupling (G. T. Hermanson, Bioconjugate Techniques, Academic Press, San Diego, 1996), producing a PAA-HAP chain.

Between 100 and 5000 □g block copolymer is then added to 100 μL of aqueous solution containing 10 μg of EPO. The weight ratio of PAA-HAP to PAA chains is between 1 to 1 and 1 to 100. EPO is bound to the peptide of the PAA-HAP chains. The aqueous solution has a pH between 5 and 7 and contains between 10 and 200 mM NaCl. Nanoparticle formation is spontaneous.

### Example 17

An aqueous phase is prepared comprised of 85 wt % water and 15 wt % IMMA (inulin multi-methacrylate). Three grams (3 g) of aqueous phase are mixed with 30 g of an oil+surfactant phase composed of 8.5 wt % Igepal CO-520 in cyclohexane. One gram of a sodium periodate solution in water is added to the mixture, so that there is a maximum of one periodate equivalent per glucose monomer unit (from the IMMA). The in-situ oxidation is allowed to proceed for ca. 10 minutes before an aldehyde-containing peptide is added. A modified version of Arg-Gly-Asp-D Phe-Lys (which contains levulinic acid on the terminal lysine) was used as the peptide. A concentrated solution (of at least 1 g/mL) of bis-[2-((2-amino-oxy)ethylamido)ethyl]amine in water is added to the microemulsion and allowed to react for an hour, crosslinking the amino-oxy moieties with the aldehydes functions.

An aqueous solution of Eosin Y, where the photoinitiator represents from 0.001 to 0.1 wt % of the IMMA mass, is added to the reverse microemulsion. The microemulsion is stirred and irradiated with a visible light source of at least 100 W for 20 min to two hours to form polymerized nanoparticles.

### Example 18

An aqueous phase is prepared comprised of 85 wt % water and 15 wt % inulin. Two grams (2 g) of aqueous phase are mixed with 30 g of an oil+surfactant phase composed of 8.5 wt % Igepal CO-520 in cyclohexane. One gram of a sodium periodate solution in water is added to the mixture, so that there is a maximum of one periodate equivalent per glucose monomer unit (from the inulin). The in-situ oxidation is allowed to proceed for ca. 10 minutes. To the microemulsion is added 1 g of an aqueous solution of interleukin-2 (from 0.1 to 5 μg) in the presence of a sufficient amount of an hexamer peptide terminated by levulinic acid (that contains an aldhehyde function) binding to interleukin-2 with an affinity constant in the micromolar to nanomolar.

A solution of bis-[2-((2-amino-oxy)ethylamido)ethyl]amine in water is added to the microemulsion and allowed to react for three hours, crosslinking the amino-oxy moieties with the aldehydes functions. The nanoparticles containing the interleukin-2/HAP complexes are purified by methods known of the art.

### Example 19

Following the method of Deming (Deming, T. J., Nature 1997, 390, 386-389; Deming, T. J., J. Am. Chem. Soc. 1997, 119, 2759-2760) poly(leucine) (Mw=2200) is prepared from the polymerization of leucine N-carboxyanhydride. In this case, the nickel polymerization catalyst is prepared with the HAP to interleukin-2 serving as the initiating ligand following the method of Curtin (Curtin, S. A., Deming, T. J., J. Am. Chem. Soc., 1999, 121, 7427-7428). In this case, the amine terminus of the interleukin-2 is protected with the allyloxycarbonyl (Alloc) protecting group, while the rest of the HAP to interleukin-2 is protected with other protecting groups commonly used in peptide synthesis. This HAP/poly(leucine) complex is then coupled to the carboxylate terminus of the protected peptide Arg-Gly-Asp-D Phe-Lys, by the action of 1,3-dicyclohexylcarbodiimide in DMF. The DMF is removed under vacuum, and the resulting solids are washed extensively with chloroform to remove the urea side product. After deprotection of the Hap to interleukin-2 and the peptide Arg-Gly-Asp-D Phe-Lys, the HAP to interleukin-2/poly(leucine)/peptide block copolymer is dissolved in DMF and added dropwise to an aqueous solution of interleukin-2. The resulting aqueous solution is dialyzed to remove DMF.

In an alternative formulation, 10-20% of the leucine of the poly(leucine) block may be substituted by lysine as a random copolymer. After synthesis of the polymer as described above, the lysine residues are deprotected. The lysine residues of the PAA block are then functionalized with levulinic acid, to include a ketone in the PAA block. The HAP to interleukin-2 and the Arg-Gly-Asp-D Phe-Lys peptide are then deprotected. Micelles are then formed as outlined above, in the presence of the interleukin-2, and di-amino-oxy functionalized PEG (Mw=5000), dissolved in DMF, is added to the water solution to crosslink the nanoparticle interior. The particles are then purified and isolated as before.

### Example 20

(Leu-Val)/Glu block copolymer chains (PAA chains), with an AB structure and with the hydrophobic block comprised of a random copolymer of Leu and Val and having a molecular weight between 1,000 to 10,000 Da, and with the hydrophilic block comprised of Glu and having a molecular weight between 5,000 and 20,000, thus with a total molecular weight of 6,000 to 30,000 Da, are formed through a controlled polymerization mechanism (supra Deming). Also, (Leu-Val)/Glu block copolymer chains (PAA chains), with an AB structure, with the hydrophobic block comprised of a random copolymer of Leu and Val and having a molecular weight of approximately 1,000 to 10,000 Da and the hydrophilic block comprised of Glu and having a molecular weight between 1,000 and 10,000 Da and thus with a total molecular weight of 2,000 and 20,000 Da, are formed through a controlled polymerization mechanism (supra Deming). This second “short” chain structure is used for peptide coupling. The acid groups of the Glu residues are kept methylated and the carboxy terminus is protected to aid in attachment of the high affinity peptide in the next step.

A peptide that binds to a soluble portion of EGFr, herein defined as “sEGFr”, preferably with an affinity constant in the micromolar to nanomolar range, is purchased. The specific peptide to be purchased is found, for example, through the screening of a peptide library. The peptide sequence used is not an agonist to EGFr. The carboxyl terminus of this peptide is attached to the amine terminus of a “short” (Leu-Val)/Glu PAA chain through EDC coupling (G. T. Hermanson, Bioconjugate Techniques, Academic Press, San Diego, 1996), producing a PAA-high affinity peptide chain.

Between 100 and 5000 μg block copolymer is then added to 100 μL of aqueous solution containing 10 μg of sEGFr. The weight ratio of PAA-peptide to PAA chains is between 1 to 1 and 1 to 100. The sEGFr is bound to the peptide of the PAA-peptide chains. The aqueous solution has a pH between 5 and 7 and contains between 10 and 200 mM NaCl. Nanoparticle formation is spontaneous.

In the present example the peptide that binds to EGFr serves as both the Type 1 and Type 2 peptide. As the Type 1 peptide, the peptide serves to bind to the sEGFr, incorporating the sEGFr into the nanoparticle. Acting as the Type 2 peptide, the peptide will serve to target tumor tissues overexpressing the EGFr receptor. The release of the therapeutic agent, that is the sEGFr, will then occur in a concentrated manner in the tumor. There, it can bind to EGF. This can reduce the binding of EGF to a tumor cell surface EGFr, and thus reduce the proliferation of tumor cells.

### Example 21

An aqueous phase is prepared comprised of 85 wt % water and 15 wt % of inulin. Two grams (2 g) of aqueous phase are mixed with 30 g of an oil+surfactant phase composed of 8.5 wt % Igepal CO-520 in cyclohexane. One gram of a sodium periodate solution in water is added to the mixture, so that there is a maximum of one periodate equivalent per glucose monomer unit (from the inulin). The in-situ oxidation is allowed to proceed for ca. 10 minutes. Erythopoietin (from 0.05 to 2 μg) is added in the presence of a sufficient amount of an heptamer peptide terminated by levulinic acid (that contains an aldehyde function) that binds to EPO with an affinity constant in the micromolar to nanomolar.

A solution of bis-[2-((2-amino-oxy)ethylamido)ethyl]amine in water is added to the microemulsion and allowed to react for three hours, crosslinking the amino-oxy moieties with the aldehydes functions. The nanoparticles containing the EPO/HAP complex are purified by methods known in the art and resuspended in aqueous solution in the presence of an excess of diamino-oxy-polyethyleneglycol. The reaction of diamino-oxy-polyethyleneglycol (with remaining aldehydes on the nanoparticles) is allowed to proceed for 12 hours. The nanoparticles are separated from the unreacted diamino-oxy-polyethyleneglycol by FPLC, coupled to the aldehyde terminus of a “targeting” peptide Arg-Gly-Asp-D Phe-Lys-levulinic acid and purified by dialysis.

What is claimed is:

1. A water-soluble polymeric nanoparticle functionalized by at least two peptide moieties covalently linked to the nanoparticle polymeric core structure, the peptide moieties possessing high affinity to biomolecules.

2. A polymeric nanoparticle according to claim 1 wherein the peptide moieties possess high affinity to therapeutic proteins.

3. A polymeric nanoparticle according to claim 1 wherein the peptide moieties possess high affinity to proteins expressed on a cell or a tissue.

4. A polymeric nanoparticle according to claim 1 comprising at least two peptide moiety possessing high affinity to therapeutic proteins and at least two peptide moiety possessing high affinity to proteins expressed on a cell or a tissue.

5. A water-soluble polymeric nanoparticle comprising i) at least two peptide moieties covalently linked to the nanoparticle polymeric core structure, each of the peptide moieties possessing high affinity to a therapeutic protein; and ii) a therapeutic protein noncovalently linked to the peptide moiety and being at least partially enclosed within the nanoparticle.

6. A water-soluble polymeric nanoparticle according to claim 5 which is further functionalized with at least two additional peptide moieties covalently linked to the nanoparticle polymeric core structure, the additional peptide moieties possessing high affinity to proteins expressed on a cell or a tissue.

7. A polymeric nanoparticle according to claim 1 wherein at least one of the peptide moieties comprises a peptide sequence selected from the group consisting of Arg-Gly-Asp-D Phe-Lys and Arg-Gly-Asp-D Phe-Lys-Cys.

8. A polymeric nanoparticle according to any of claims 1 to 7 wherein the polymeric core comprises a hydrophobic/hydrophilic block copolymer.

9. A polymeric nanoparticle according to claim 8 wherein the block copolymer comprises poly(amino acid).

10. A polymeric nanoparticle according to claim 9 wherein the poly(amino acid) comprises a hydrophobic block composed of hydrophobic or neutral amino acids, and random copolymers of any two of these; and a hydrophilic block composed of hydrophilic amino acids.

11. A polymeric nanoparticle according to claim 8 wherein the block copolymer comprises a poly(amino acid) hydrophobic block and a polyethylene glycol hydrophilic block.

12. A polymeric nanoparticle according to claim 8 wherein the block copolymer comprises a polycaprolactone hydrophobic block and a polyethylene glycol hydrophilic block.

13. A polymeric nanoparticle according to any of claims 1 to 7 wherein the polymeric core comprises a crosslinked hydrophilic polymer.

14. A polymeric nanoparticle according to claim 13 wherein the hydrophilic polymer comprises crosslinked hydrophilic building blocks, at least some of the building blocks being carbohydrates.

15. A polymeric nanoparticle according to any of claims 1 to 14 characterized by having a high water content.

16. A polymeric nanoparticle according to any of claims 1 to 15 which further comprises at least one enhancer molecule covalently attached to the nanoparticle polymeric core structure.

17. A polymeric nanoparticle according to any of claim 1-16 which further comprises at least one polyethylene glycol molecule covalently attached to the nanoparticle polymeric core structure.

18. A polymeric nanoparticle according to any of claims 1 to 17 wherein the peptide moiety is covalently linked directly to a polymer molecule.

19. A polymeric nanoparticle according to any of claims 1 to 17 wherein the peptide moiety is covalently linked to a polymer molecule by a linker molecule.

20. A polymeric nanoparticle according to claim 19 wherein the linker molecule is a polyethylene glycol chain.

21. A method for the molecular recognition of a biomolecular target, the method comprising exposing the biomolecular target to a water-soluble polymeric nanoparticle functionalized by at least two peptide moieties covalently linked to the nanoparticle polymeric core structure, at least one of the peptide moieties possessing high affinity to proteins expressed on the biomolecular target.

22. A method for controllably releasing a therapeutic protein to an environment in a mammalian body, the method comprising administering to the environment a water-soluble polymeric nanoparticle comprising i) at least two peptide moieties covalently linked to the nanoparticle polymeric core structure, each of the peptide moieties possessing high affinity to the therapeutic protein; and ii) the therapeutic protein noncovalently linked to the peptide moiety and being at least partially enclosed within the nanoparticle.

23. A method for the controlled delivery of a therapeutic protein to the vicinity of a targeted cell or tissue type, the method comprising administering to an environment containing the targeted cell or tissue type, a water-soluble polymeric nanoparticle comprising i) at least two first peptide moieties covalently linked to the nanoparticle polymeric core structure, each of the first peptide moieties possessing high affinity to the targeted cell or tissue type; ii) at least two second peptide moieties covalently linked to the nanoparticle polymeric core structure, each of the second peptide moieties possessing high affinity to the therapeutic protein; and iii) the therapeutic protein noncovalently linked to the second peptide moiety and being at least partially enclosed within the nanoparticle.

24. A method for synthesizing a water-soluble peptide-functionalized polymeric nanoparticle, the method comprising adding an aqueous phase containing hydrophilic building blocks, the building blocks comprising hydrophilic monomers with crosslinkable groups, to an organic solvent comprising at least one surfactant;
reacting the crosslinkable groups of the building blocks to covalently crosslink the building blocks to give a hydrophilic polymeric nanoparticle;
removing surfactant and the organic solvent;
adding peptide moieties to a solution containing the nanoparticle, the peptide moieties comprising a functionality for attachment to the nanoparticle and the peptide moieties possessing high affinity to proteins expressed on a cell or a tissue; and
reacting the peptide moieties and the nanoparticle to covalently bond the peptide and nanoparticle.

25. A method according to claim 24 wherein the aqueous phase further comprises therapeutic proteins and peptide moieties possessing high affinity to the therapeutic proteins.

26. A method according to claim 24 or 25 wherein at least some of the hydrophilic building blocks comprise carbohydrates.
